(19) United States
(12) Patent Application Publication
(10) Pub. No.: US 2026/0080999 A1
(43) Pub. Date: Mar. 19, 2026

(54) HEALTH MANAGEMENT SYSTEM

(71) Applicant: Ahee Build Health, Inc., Nottingham, NH (US)

(72) Inventors: J. Christopher Flaherty, Nottingham, NH (US); R. Maxwell Flaherty, Topsfield, MA (US)

(21) Appl. No.: 19/192,843

(22) Filed: Apr. 29, 2025

Related U.S. Application Data
(63) Continuation of application No. 17/299,433, now abandoned.
(60) Provisional application No. 62/774,954, filed on Dec. 4, 2018.

(51) Int. Cl. G16H 20/60; G16H 10/60; G16H 50/20

(52) U.S. Cl. CPC G16H 20/60; G16H 10/60; G16H 50/20

(57) ABSTRACT

A system for providing food product to a patient. The system comprises a patient data device and a processing unit. The patient data device comprises a user interface, and is configured to receive information comprising a food product request from the patient. The processing unit is configured to receive information from the patient data device. The processing unit comprises a memory module and an algorithm. The memory module is configured to store at least: patient information, and food product information. The algorithm is configured to identify a food product for the patient based on: the food product request, the patient information, and the food product information. Methods of providing food product to a patient are also disclosed.

### CROSS-REFERENCE TO RELATED APPLICATIONS

This application is a continuation application of U.S. patent application Serial No. 17/299,433, entitled “Health Management System”, filed on Jun. 3, 2021, Publication Number US2022/0084654, published Mar. 17, 2022, which is a U.S. National Stage entry of International Patent Application No.: PCT/US2019/064394, entitled “Health Management System”, filed Dec. 4, 2019, Publication Number WO 2020/117897, published Jun. 11, 2020, which application claims priority to U.S. Provisional Application Ser. No. 62/774,954, entitled “Health Management System”, filed Dec. 4, 2018, the content of which is incorporated herein by reference in its entirety for all purposes.

### FIELD OF THE INVENTION

The present inventive concepts relate generally to systems for users to improve and/or maintain the health of themselves and/or others, and in particular, to systems that aid in the ingestion of personalized food products that provide health benefits to particular users.

### BACKGROUND

There is a popular belief that proper nutrition can help promote good health and potentially even treat various medical conditions. There are a number of products being promoted as providing health benefits, but without scientific evidence (e.g. not evidence readily available to the consumer). There is also a concern that a product that may provide health benefits to some people, may not provide a benefit to others, and may actually be detrimental. Availability of healthy products under certain conditions (e.g. traveling) is also a concern. Very often people that become initially interested in learning about nutrition and eating healthily, become quickly frustrated by confusing and conflicting information available to them.

There is a need for systems that allow one or more users to easily identify, procure, prepare, and/or ingest food products that allow the users to maintain and/or improve their health.

### SUMMARY

According to one aspect of the present inventive concepts, a system for providing food product to a patient comprises a patient data device and a processing unit. The patient data device comprises a user interface and is configured to receive information comprising a food product request from the patient. The processing unit is configured to receive information from the patient data device. The processing unit comprises a memory module and an algorithm. The memory module is configured to store at least: patient information and food product information. The algorithm is configured to identify a food product for the patient based on: the food product request, the patient information, and the food product information. The algorithm can be further configured to identify the food product based on other information as well.

In some embodiments, the system is configured to identify a food product that tends to improve and/or maintain the health of the patient.

In some embodiments, the system is configured to deliver the food product to the patient.

In some embodiments, the system is configured to cause the food product to be delivered to the patient.

In some embodiments, the patient comprises a healthy human.

In some embodiments, the patient is afflicted with one or more undesired medical conditions.

In some embodiments, the patient comprises a group of people. The group of people can comprise a family.

In some embodiments, the food product comprises food product data related to one or more food products identified by the algorithm. The food product data can comprise information selected from the group consisting of: caloric information; sugar information; carbohydrate information; fat information; trans fat information; protein information; vitamin information; and combinations thereof. The food product data can comprise a recipe for a meal. The food product data can comprise a health score of the one or more food products identified by the algorithm.

In some embodiments, the food product comprises one or more ingestible food products.

In some embodiments, the food product comprises multiple ingredients to be used to prepare a meal.

In some embodiments, the food product request comprises a request for a food product previously identified by the algorithm.

In some embodiments, the food product request comprises a request for a particular size of meal.

In some embodiments, the food product request comprises a request for a healthier alternative to a particular food.

In some embodiments, the patient information comprises information entered into the system by the patient.

In some embodiments, the patient information comprises information entered into the system by a clinician.

In some embodiments, the patient information comprises patient health information. The patient health information can comprise information selected from the group consisting of: medical condition information, such as known or suspected presence of one or more medical conditions, such as heart disease, neurological disease, Alzheimer's disease, Crohn's disease, celiac disease, diabetes, fatty liver, polycystic ovarian syndrome, and/or other diseases or disorders; blood information, such as blood component level information; cholesterol information; testosterone information; estrogen level; biomarker level; urine information; biopsy information; histology information; blood flow information, such as restricted artery information; bone information, such as osteoporosis information; genetic information; genetic predisposition information; vitamin and/or mineral level information; sleep apnea information; allergy information; and combinations thereof. The patient health information can comprise genetic information. The genetic information can comprise information received from a DNA testing company.

In some embodiments, the patient information comprises patient preference information.

In some embodiments, the patient information comprises patient location information.

In some embodiments, the patient information comprises patient pantry information.

In some embodiments, the patient information comprises patient goal information.

In some embodiments, the patient information comprises patient appetite level information.

In some embodiments, the patient information comprises patient fear information.

In some embodiments, the patient information comprises recent patient history data.

In some embodiments, the patient information comprises patient medication information.

In some embodiments, the patient information comprises patient clinical procedure information.

In some embodiments, the food product information comprises information provided by a supplier of the food product.

In some embodiments, the food product information comprises information provided by a clinician.

In some embodiments, the food product information comprises a health score related to the food product.

In some embodiments, the algorithm is configured to provide a healthier alternative to the food product requested.

In some embodiments, the algorithm is configured to provide a list of multiple food product options. The system can be configured to provide the food product based on a patient selection from the list.

In some embodiments, the algorithm is further configured to identify the food product based on generic clinical data.

In some embodiments, the algorithm is further configured to identify the food product based on supplier data.

In some embodiments, the algorithm is further configured to identify the food product based on other data. The other data can comprise food product transportation data.

In some embodiments, the algorithm is configured to identify the food product based on patient medication information of the patient.

In some embodiments, the algorithm is configured to identify the food product based on at least one of: an allergy of the patient; a food sensitivity of the patient; or a food intolerance of the patient.

In some embodiments, the system can further comprise at least one sensor, and the algorithm is configured to identify the food product based on data recorded by the at least one sensor.

In some embodiments, the algorithm is configured to identify the food product based on recent history information of the patient. The recent history information can comprise information about food recently ingested by the patient.

In some embodiments, the algorithm is configured to identify the food product based on system requested recent history information of the patient.

In some embodiments, the algorithm is configured to identify the food product based on system estimated recent history information of the patient.

In some embodiments, the algorithm is configured to identify the food product based on patient preference information.

In some embodiments, the algorithm is configured to identify the food product based on monitored public data.

In some embodiments, the algorithm is configured to identify the food product based on a diet plan.

In some embodiments, the algorithm is configured to identify a food product that includes a system-recommended supplement.

In some embodiments, the algorithm is configured to identify a food product that includes a replacement food product.

In some embodiments, the algorithm is configured to identify a food product that includes a requested food product and an additional food product.

In some embodiments, the algorithm is configured to identify a food product that includes food product data.

In some embodiments, the patient data device comprises a first patient data device and a second patient data device. The first patient data device and the second patient data device can be used by the patient. The first patient data device can be used by the patient, and the second patient data device can be used by an additional patient.

In some embodiments, the patient data device comprises at least a portion of the processing unit.

In some embodiments, the system can further comprise a patient diagnostic device configured to provide diagnostic data of the patient to the processing unit. The algorithm can be further configured to identify the food product based on the provided diagnostic data. The patient diagnostic device can be configured to provide diagnostic information selected from the group consisting of: activity level information; motion information; blood glucose information; blood pressure information; heart rate information; respiration information; pH information; digestive information; sleep information; sleep apnea information; and combinations thereof. The system can be configured to determine if a food product was ingested by the patient based on the provided diagnostic data.

In some embodiments, the system can further comprise a patient therapy device configured to perform a therapeutic event on the patient. The patient therapy device can be further configured to provide therapeutic data to the processing unit. The algorithm can be further configured to identify the food product based on the therapeutic data. The patient therapy device can comprise a device selected from the group consisting of: drug delivery device; insulin delivery device; external device; implanted device; pacemaker; defibrillator; a drug; pain control device; stimulator; implanted and/or external stimulator; and combinations thereof.

In some embodiments, the system can further comprise a clinician data device configured to allow a clinician to enter information into the system. The algorithm can be further configured to identify the food product based on the clinician entered information.

In some embodiments, the system can further comprise a supplier data device configured to allow a supplier to enter information into the system. The algorithm can be further configured to identify the food product based on the supplier entered information.

In some embodiments, the system can further comprise a system manufacturer data device configured to allow a manufacturer of the system to enter information into the system. The algorithm can be further configured to identify the food product based on the system manufacturer entered information.

In some embodiments, the system can further comprise a network configured to operably connect multiple components of the system for information transfer between the multiple components. The network can comprise a network selected from the group consisting of: the internet; a private computer network; a cellular network; a wired network; a wireless network; another information-transmitting network; and combinations thereof.

In some embodiments, the system can further comprise one or more functional elements. The one or more functional elements can comprise one or more sensors. The one or more functional elements can comprise one or more transducers. The one or more transducers can comprise at least one transducer configured to alert the patient. The one or more functional elements can comprise an observational device.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of a system for providing food product to a patient, consistent with the present inventive concepts.

FIG. 2 illustrates a flow chart of a method for a patient to obtain a food product, consistent with the present inventive concepts.

FIG. 3 illustrates a flow chart of a method for a patient to obtain a food product based on a medication regimen of the patient, consistent with the present inventive concepts.

FIG. 4 illustrates a flow chart of a method for a patient to obtain a food product based on an allergy of the patient, consistent with the present inventive concepts.

FIG. 5 illustrates a flow chart of a method for a patient to obtain a food product based on sensor data, consistent with the present inventive concepts.

FIG. 6 illustrates a flow chart of a method for a patient to obtain a food product based on recent patient history, consistent with the present inventive concepts.

FIG. 7 illustrates a flow chart of a method for a patient to obtain a food product based on system requested recent patient history, consistent with the present inventive concepts.

FIG. 8 illustrates a flow chart of a method for a patient to obtain a food product based on system estimated recent patient history, consistent with the present inventive concepts.

FIG. 9 illustrates a flow chart of a method for a patient to obtain a food product based on a patient preference, consistent with the present inventive concepts.

FIG. 10 illustrates a flow chart of a method for a patient to obtain a food product based on monitored public data, consistent with the present inventive concepts.

FIG. 11 illustrates a flow chart of a method for a patient to obtain a food product based on a diet plan, consistent with the present inventive concepts.

FIG. 12 illustrates a flow chart of a method for a patient to obtain a food product that includes a system-recommended supplement, consistent with the present inventive concepts.

FIG. 13 illustrates a flow chart of a method for a patient to obtain a replacement food product, consistent with the present inventive concepts.

FIG. 14 illustrates a flow chart of a method for a patient to obtain a requested food product and an additional food product, consistent with the present inventive concepts.

FIG. 15 illustrates a flow chart of a method for a patient to obtain food product data regarding a specific food product, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,”etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location.

For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention”shall include the acts of “reduce”, “reducing”, and “reduction”, respectively.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

The terms “data”and “information”are used interchangeably.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first quantity such as to provide a particular therapeutic benefit and/or other benefit to a user) and below a second threshold (e.g. below a second quantity, greater than the first, such as to avoid causing an undesired and/or unnecessary event). In some embodiments, a threshold value is determined to include a safety margin, such as to account for patient variability, system variability, tolerances, and the like. As used herein, “exceeding a threshold” can relate to a parameter going above a maximum threshold, going below a minimum threshold, existing within a range of threshold values, and/or existing outside of a range of threshold values.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a patient height, weight, and/or body mass parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver a therapeutic agent). In some embodiments, a functional element comprises one or more elements constructed and arranged to perform a function selected from the group consisting of: deliver energy; extract energy (e.g. to cool a component); deliver a drug or other agent; manipulate a system component; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise one or more functional elements.

The term “transducer” where used herein is to be taken to include any component or combination of components that receives energy or any input, and produces an output. In some configurations, a transducer converts an electrical signal into any output, such as light (e.g. a transducer comprising a light emitting diode or light bulb), sound (e.g. a transducer comprising a piezo crystal configured to deliver ultrasound energy), pressure, heat energy, cryogenic energy, chemical energy; mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal.

As used herein, the term “patient” shall include one or more human subjects that may be relatively healthy, and/or one or more human subjects that have one or more undesired medical conditions. Each patient may be an individual that wishes to ingest food products to prevent disease or otherwise maintain a healthy state. Alternatively or additionally, the patient may be an individual that wants to achieve an improvement (e.g. a self-improvement), such as a cure, elimination, and/or at least a reduction in magnitude of one or more undesired medical conditions and/or other undesired conditions (e.g. an undesired habit). Alternatively or additionally, the patient may be one or more individuals that provide food products to a group, such as a head of a household that provides food to a family, and/or a cafeteria management person that provides food to a group (e.g. a group of students, a group of patients in a hospital, and the like).

As used herein, the term “medical condition” and its derivatives shall include one or more diseases, disorders, and/or other medical conditions (e.g. undesired medical conditions) of a patient.

As used herein, the term “allergy” and its derivatives shall refer not only to one or more allergies, but also to food sensitivities, food intolerances, and/or other adverse reactions to one or more particular types of food.

As used herein, the term “data device” and its derivatives shall include a component that allows a user to enter and/or receive information. A data device can comprise one or more user input components and/or one or more user output components, such as to allow a user to enter information and/or receive information. User input components include but are not limited to: a keyboard; a keypad; a touch screen; a mouse; a joystick; a microphone; a camera (e.g. camera configured to record patient cues); and combinations of these. User output components include but are not limited to: a display; a speaker; an indicator light; a tactile transducer; and combinations of these. Data devices of the present inventive concepts can comprise a device selected from the group consisting of: handheld electronic device; a phone (e.g. a smartphone or other cell phone); wristwatch (e.g. a smart watch); tablet; laptop computer; desktop computer; an artificial intelligence (AI) assistant device (e.g. an Alexa, Siri, Google Assistant, or Cortana device); and combinations of one, two, or more of these.

As used herein, the term “food product” and its derivatives shall include one or more ingestible substances, including discrete items (“ingredients”) and combinations of ingredients (e.g. cooked ingredients, ingredients mixed together, and/or ingredients provided as a set). Food product shall include prepared foods, snacks, and entire meals. As used herein, food product shall also include “health agents” as defined herein. Food products can include ingredients and/or prepared meals that are provided in a restaurant and/or by a commercial food delivery service (e.g. a food product delivery service). A food product can include a “neutralizing agent”configured to reduce adverse effects of another ingested item.

As used herein, the term “algorithm” shall include a mathematical or other process in which quantitative, qualitative, and/or other data is analyzed to produce a result. The algorithm can include in its analysis databases of data. When an algorithm is “based on” one or more parameters, it shall be deemed based on at least those one or more parameters (e.g. the algorithm can be additionally based on other parameters). In some embodiments, an algorithm can include a “bias” (e.g. a “biased algorithm”) such as to tend to produce one particular result versus another.

As used herein, the term “health agent” shall include a substance that is believed to provide a health benefit to a particular patient (e.g. a particular one or more patients). The health agent can be administered orally, intravascularly, via injection, via suppository, via transdermal drug delivery, and/or via other means in which an agent can be delivered systemically or locally to the patient. Health agents shall include but are not limited to: pharmaceutical drugs; nutraceuticals; vitamins; minerals; probiotics; supplements; and the like. Health agents shall include food products that include a health agent. A health agent shall include one, two, or more health agents.

As used herein, the term “patient medication information” and its derivatives shall include data related to pharmaceutical drugs, vitamins, minerals, supplements, nutraceuticals, and/or other health agents administered to the patient (e.g. a medication regimen of the patient), such as to prevent and/or treat a medical condition of the patient. Patient medication information shall include data related to a health agent (e.g. one or more health agents) administered to the patient in the past, in the present (currently), and/or in the future; and can include quantity and/or temporal information related to the administration of the health agent (e.g. XX grams/day for the past N days).

As used herein, the term “health score” shall include a quantitative or qualitative assessment used to characterize a food product's impact on the health of a patient (e.g. a known and/or potential effect on the health of a patient). In some embodiments, a health score is an assessment of a food product's impact (e.g. known or potential impact) on a particular condition of the patient. For example, a food product can be assigned a health score that represents it being beneficial (e.g. potentially beneficial) to one or more medical conditions of the patient (e.g. a high number, multiple gold stars, and the like). Conversely, a food product can be assigned a health score that represents it being detrimental to one or more medical conditions of the patient (e.g. a negative number, multiple thumbs down, and the like). In some embodiments, a food product may get a first health score for a first medical condition of a patient (e.g. a score related to the patient's diabetes), and a second, potentially different health score, for a second medical condition of the patient (e.g. a score related to the patient's arthritis).

As used herein, the term “substitute food product” shall include a food product that is similar to another food product (e.g. similar in taste to a requested food product). In some embodiments, a substitute food product includes a food product that is similar in taste to a different food product, but has a more desirable health score for a particular patient.

As used herein, the term “patient activity data” shall include information related to the patient's past, present, and/or future (e.g. predicted) state of activity.

As used herein, the term “patient wellness data” shall include information related to the patient's past, present, and/or future (e.g. predicted) state of health.

As used herein, the term “recent patient history data” shall include data related to recent patient activity (e.g. recently ingested food product, recent physical activity, and the like) and/or recent patient diagnostic information obtained (e.g. diagnostic information recorded by a patient-carried and/or patient-implanted diagnostic device). Recent patient history data can include recent patient activity data and/or recent patient wellness data. As used herein, recent patient history data can include data representing a duration of time between the present time and a previous time. For example, the duration of time associated with “recent patient history data” can comprise a duration of no more than: 5 minutes, 15 minutes, 30 minutes, 1 hour, 4 hours, 8 hours, 12 hours, 1 day, 2 days, 3 days, 1 week, 2 weeks, 1 month, 3 months, 6 months, and/or 1 year. Alternatively or additionally, the duration of time can comprise a duration of at least: 5 minutes, 15 minutes, 30 minutes, 1 hour, 4 hours, 8 hours, 12 hours, 1 day, 2 days, 3 days, 1 week, 2 weeks, 1 month, 3 months, 6 months, and/or 1 year.

As used herein, the term “food product parameter” and its derivatives shall include one or more parameters associated with a particular food product, such as a parameter selected from the group consisting of: an ingredient of the food product; calories associated with ingestion of the food product; a level of an ingredient of the food product such as a level of a vitamin, a mineral, a fat, and/or a toxin; one or more health scores of the food product; the cost of the food product; the availability of the food product; a supplier of the food product; and combinations of one, two, or more of these.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to perform any of the steps, including but not limited to: displaying, communicating with the user, analyzing, modifying parameters (including timing, frequency, intensity, etc.), determining, alerting, and/or the like.

The systems of the present inventive concepts include data devices that allow a patient, or other user of the system, to enter a request related to a meal or other food product, such as a food product to be ingested by a patient (e.g. the same patient or one or more other patients). The system can provide one or more food products, and/or information regarding one or more food products, based on various information available to the system (e.g. information input into the system), such as patient information (e.g. patient health information) and/or other information. The system can include a memory module configured to store at least patient information and food product information. The system can include one or more algorithms configured to identify a food product for the patient based on: a food product request; patient information; and/or food product information.

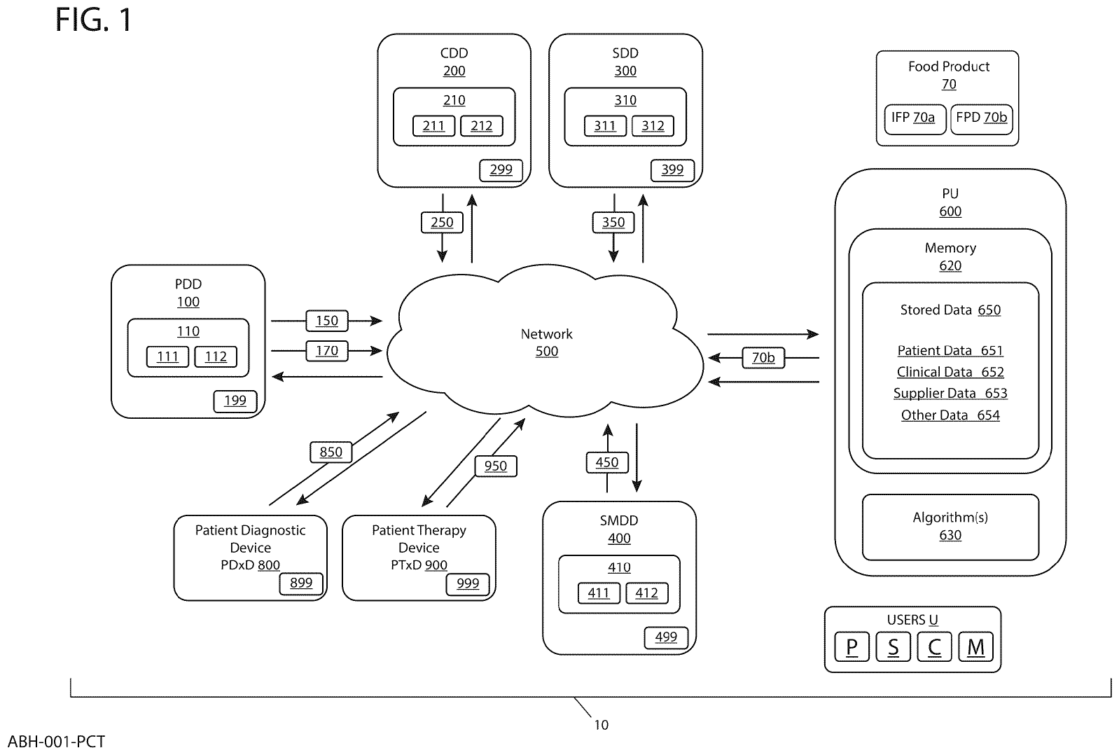

Referring now to FIG. 1, a schematic view of a system for providing a food product to a patient is illustrated, consistent with the present inventive concepts. System 10 is configured to interface with one or more users, user U, such as one or more patients (user P herein), as well as other users of system 10, such as are defined herein. System 10 includes a patient data device, PDD 100, used by user P to obtain FP 70, where FP 70 comprises one or more food products and/or food product data. System 10 provides FP 70 based on a request of user P, a food product request, FPR 170. FP 70 can include a full meal or simply any other ingestible food product, IFP 70a, and/or simply data related to a food product, FPD 70b (e.g. information which can be used by user P to purchase, create, and/or otherwise acquire FP 70). In some embodiments, FP 70 includes both IFP 70a as well as FPD 70b. The FP 70 identified, listed, recommended, suggested, described, delivered, prepared, and/or otherwise provided (“provided” herein) by system 10 can be provided based on information provided by user P, patient provided data 150, and/or it can be provided based on other information, as described herein. System 10 can be configured to deliver FP 70 to user P, and/or it can be configured to cause FP 70 to be delivered to user P (e.g. via a food delivery service).

In addition to user P, user U can include other users, such as user S, user C, and/or user M, each as defined herein.

User P and/or other users U of system 10 can interface with one or more data devices, as described herein, to provide and/or receive information to and/or from system 10. System 10 can include a processing unit, PU 600, which receives and stores various data, stored data 650, from the various data devices. Stored data 650 can be stored in a memory module, memory 620. Stored data 650 can comprise one or more databases of information, such as: a patient information database, patient data 651; a generic clinical information database, generic clinical data 652; a supplier information database, supplier data 653; and/or a database of other information, other data 654; each described herein. Stored data 650 can comprise a list of food products, such as a list of products to be analyzed and potentially identified by algorithm 630 as FP 70. Stored data 650 can comprise one or more food product requests, FPR 170, such as a library of previously entered FPRs 170. In some embodiments, stored data 650 includes a chronology of activity related to the use of system 10 by user P, such as a chronology of FPRs 170, a list of FPs 70 suggested (e.g. a list of FPD 70b identified by algorithm 630), a list of FPs 70 obtained and/or ingested by user P, and/or other information. PU 600 can comprise one or more algorithms, algorithm 630, which can be configured to analyze and/or otherwise process stored data 650, such as to choose, determine, estimate, and/or otherwise identify (“identify” herein) FP 70 (e.g. FPD 70b) and/or to provide other information to a user U, as described in detail herein.

System 10 includes a computer and/or other information sharing network, network 500, which operably connects multiple data devices and/or other components of system 10 for information transmissions between components (e.g. wired or wireless transmissions). Network 500 can comprise the internet, a private computer network, a cellular network, a wired network, a wireless network, and/or other information-transmitting network.

System 10 can include various data devices, such as: PDD 100; a clinician data device, CDD 200; a supplier data device, SDD 300; a system 10 manufacturer data device, SMDD 400; a patient diagnostic device, PDxD 800; and/or a patient therapeutic device, PTxD 900; each as described herein.

PDD 100 can receive information from user P that is transmitted to PU 600 and then included in stored data 650 (e.g. as patient data 651 and/or other data 654). Alternatively or additionally, information received from user P by PDD 100 can be transmitted to another component of system 10, such as when transmitted to CDD 200, SDD, 300, SMDD 400, PDxD 800, and/or PTxD 900. PDD 100 can provide information to user P, such as information received from PU 600 (e.g. FPD 70b or other information), and/or information received from another component of system 10, such as information received from CDD 200, SDD 300, SMDD 400, PU 600, PDxD 800, and/or PTxD 900.

In some embodiments, system 10 includes CDD 200 which comprises one or more data devices, each of which is configured to allow one or more users C to enter and/or receive information from system 10 (e.g. via network 500). In some embodiments, a user C using CDD 200 comprises a clinician of user P (e.g. a primary care clinician, nutritional advisor, and/or other healthcare provider of user P). Alternatively or additionally, a clinician using CDD 200 can comprise a clinician that is employed with (e.g. as defined herein) the manufacturer of system 10 (e.g. a clinician-based user M, such as a clinician used to assess a user P clinical condition and/or to assess an FP 70). Using CDD 200, a user C can provide information to system 10, clinician provided data 250, such as information which is then transmitted to PU 600 and then included in stored data 650 (e.g. stored as patient data 651, generic clinical data 652, and/or other data 654). In some embodiments, CDD 200 transmits and/or receives information to and/or from another component of system 10, such as information transmitted to and/or received from PDD 100, SDD 300, SMDD 400, PU 600, PDxD 800, and/or PTxD 900.

In some embodiments, system 10 includes SDD 300 which comprises one or more data devices, each of which is configured to allow one or more users S to enter and/or receive information from system 10 (e.g. via network 500). In some embodiments, a user S using SDD 300 comprises a supplier of FP 70 (e.g. an employee of a supplier of FP 70). Using SDD 300, a user S can provide information to system 10, supplier provided data 350, such as information which is transmitted to PU 600 and then included in stored data 650 (e.g. stored as supplier data 653, FPD 70b, and/or other data 654). In some embodiments, SDD 300 transmits and/or receives information to and/or from another component of system 10, such as information transmitted to and/or received from PDD 100, CDD 200, SMDD 400, PU 600, PDxD 800, and/or PTxD 900.

In some embodiments, system 10 includes SMDD 400 which comprises one or more data devices, each of which is configured to allow one or more users M to enter and/or receive information from system 10 (e.g. via network 500). Each user M can provide information to system 10, system manufacturer provided data 450, such as information which is transmitted to PU 600 and then included in stored data 650 (e.g. stored as patient data 651, generic clinical data 652, FPD 70b, and/or other data 654). In some embodiments, SMDD 400 transmits and/or receives information to and/or from another component of system 10, such as information transmitted to and/or received from PDD 100, CDD 200, SDD 300, PU 600, PDxD 800, and/or PTxD 900.

In some embodiments, system 10 includes a patient diagnostic device, PDxD 800, which is configured to perform a diagnostic test and collect diagnostic data of user P, as described herein. PDxD 800 can comprise one or more diagnostic devices, each of which is configured to perform one or more diagnostic tests, and to transmit diagnostic device-provided data 850 to system 10, such as diagnostic information which is transmitted to PU 600 and then included in stored data 650 (e.g. stored as patient data 651, and/or other data 654). Algorithm 630 can be configured to identify FP 70 based on this diagnostic information. In some embodiments, PDxD 800 transmits and/or receives information to and/or from another component of system 10, such as information transmitted to and/or received from PDD 100, CDD 200, SDD 300, SMDD 400, PU 600, and/or PTxD 900.

In some embodiments, system 10 includes a patient therapeutic device, PTxD 900, which can be configured to perform a therapeutic procedure on user P, as described herein. PTxD 900 can comprise one or more therapeutic devices, each of which is configured to perform one or more therapies (e.g. one or more therapies performed on user P), and to transmit therapeutic device-provided data 950 to system 10, such as therapy information which is transmitted to PU 600 and then included in stored data 650 (e.g. stored as patient data 651, and/or other data 654). Algorithm 630 can be configured to identify FP 70 based on this therapeutic information. In some embodiments, PTxD 900 transmits and/or receives information to and/or from another component of system 10, such as information transmitted to and/or received from PDD 100, CDD 200, SDD 300, SMDD 400, PU 600, and/or PDxD 800.

User U can comprise one or more users selected from the group consisting of: user P; user C; user S; user M; a family member of user P; a parent of user P; a clinician; a surgeon; a nurse; a psychologist; a health care provider; an insurance company; Medicare or Medicaid; and combinations of one, two, or more of these.

User P can comprise one or more individuals which utilize system 10 to obtain IFP 70a, FPD 70b, and/or other FP 70. User P can comprise a family member (e.g. a parent) of one or more patients (e.g. other users P) that receive FP 70 (e.g. a group of children or other family members). User P can comprise one or more healthy and/or non-healthy individuals. User P can be an athlete, such as an athlete that uses system 10 to adjust their food product ingestion based on their activity level (e.g. to accommodate for seasonal variations). User P can be a person with diabetes, such as a person that uses system 10 to closely monitor: glucose levels, insulin taken, and FP 70 ingested. User P can be a pre-natal and/or post-natal woman, such as a woman that uses system 10 to identify FP 70 to ingest to improve the health of themselves and the fetus and/or resultant offspring. User P can be a person under 18 years of age, or under 13 years of age, such as when User U further comprises a parent or other guardian that prepares or at least chooses FP 70 to be ingested by the user P.

User C can comprise one or more clinicians or other healthcare professionals. User C can comprise one or more groups of healthcare professionals (e.g. medical doctors, nurses, nutritionists, therapists, and/or other healthcare professionals). User C can include one or more individuals which provide information related to user P and/or information related to FP 70. User C can be a primary care or other clinician of user P.

User S can comprise one or more suppliers of FP 70. In some embodiments, user S comprises one or more entities which provide information regarding an FP 70. User S can comprise an organization that provides for the shipping of an FP 70 (e.g. a post office, FedEx, UPS, and the like). User S can comprise an organization in the relative vicinity of user P (e.g. in the home location of user P and/or a location in which user P is currently traveling).

User M can comprise one or more personnel (“employee” herein) that is employed, contracted by, working on behalf of, and/or otherwise associated with (“employed” herein) the provider and/or manufacturer (“manufacturer” herein) of system 10. User M can comprise an owner (e.g. a partial owner or stockholder) of the manufacturer of system 10. User M can comprise one or more clinicians, nutritionists and/or dieticians (“nutritionists” herein), data analyzers, mathematicians, statisticians, and/or other users of system 10, such as users that provide and/or analyze data related to user P, FP 70, generic health information, generic food product information, and/or other information.

As described herein, system 10 can be configured to provide various forms of food product FP 70. FP 70 can comprise ingestible food product, IFP 70a, and/or food product data, FPD 70b. In some embodiments, user P can comprise a patient that ingests food product (e.g. FP 70) that is provided based on FPD 70b. FP 70 can comprise ingestible food product IFP 70a or food product data FPD 70b that comprises or represents, respectively, a single meal or multiple meals. FP 70 can comprise ingestible food product IFP 70a and/or food product data FPD 70b that comprises or represents, respectively, a desired portion size of a food product to be ingested by one or more users P. FP 70 can comprise ingestible food product IFP 70a and/or food product data FPD 70b that comprises or represents, respectively, a vitamin, mineral, supplement, and/or probiotic.

IFP 70a can comprise food product that is ingested by a user P at any time, such as in a single serving and/or in multiple servings. IFP 70a can comprise a food product that is cooked. Alternatively or additionally, IFP 70a can comprise a food product that is raw (e.g. not cooked, for ingestion raw or to be cooked at a later time).

FPD 70b can comprise data related to one or more ingredients for a meal. FPD 70b can comprise a recipe for a meal, such as a recipe including cooking instructions that are provided in written, audio, and/or video format. FPD 70b can comprise a description of an IFP 70a to be purchased (e.g. at a grocery store and/or restaurant). FPD 70b can include nutritional information for a food product, such as information selected from the group consisting of: caloric information; sugar information; carbohydrate information; fat information; trans fat information; protein information; vitamin information; and combinations of one, two, or more of these. FPD 70b can comprise a health score, as described herein, related to a food product. System 10 is configured to allow a user P to make a food product request, FPR 170. An FPR 170 can be a request for a food product to be ingested, IFP 70a, and/or data related to a food product, FPD 70b. FPR 170 can comprise a request for a specific (entire) type of meal, such as a breakfast, lunch, dinner, and/or other meal (e.g. a snack). FPR 170 can include a request for a particular size of meal, such as small, medium, or large. FPR 170 can include a request for a specific ingredient and/or specific nutritional content, such as a specific vitamin, protein, and/or vegetable, and FRP 170 can include a request for a specific amount of that specific ingredient and/or specific nutritional content. FPR 170 can comprise a request for an FP 70 previously provided by system 10, and/or a request for an FP 70 that is similar to a previously provided (e.g. previously identified) FP 70. FPR 170 can comprise multiple requests, such as a first request for one or more FPs 70, and a second request for one or more FPs 70 (e.g. multiple requests that are combined into a single request or maintained as separate requests). FPR 170 can include a request for a particular food category or other classification of an FP 70 to be provided, such as a request selected from the group consisting of: a milkshake; a healthier choice than a milkshake; a food similar to “XXXX” but healthier; a small meal; and/or a large meal.

In some embodiments, PDD 100 is configured to provide a list of multiple FPs 70, such as an options list provided in a selectable arrangement (e.g. similar to a menu), from which a user P can select one or more of the listed FPs 70.

As described herein, system 10 can include one or more devices, PDD 100, that allow user P to enter information into, and/or receive information from, system 10,

PDD 100 can comprise a data device as defined herein. PDD 100 comprises user interface 110, which can include various user input components 111 and/or user output components 112, also as defined herein.

PDD 100 can comprise one or more functional elements, such as functional element 199 shown and described herein.

PDD 100 allows user P or other user U of system 10 to enter information that is to be used and/or stored by system 10. Entered information can include patient information 150, an FPR 170, and/or other information. Information can be entered via user interface 110, such as via a keyboard of input components 111, a selectable icon or provided text (e.g. via a mouse or touch screen selection), and/or via a microphone component of user input components 111. User interface 110 can provide an “options list” (e.g. a table of selectable values), such as an FP 70 options list, in the form of text lists, graphics, icons, and the like, such as to allow user P to select an option (e.g. for user P to make an FPR 170). Alternatively or additionally, information can be input into system 10 via a microphone, a keyboard, a touch screen, and/or other input component (e.g. a component of user interface 110 or other component of system 10).

In some embodiments, multiple users P use a single PDD 100. Alternatively or additionally, a single user P can use multiple PDDs 100.

In some embodiments, system 10 includes CDD 200, which can comprise one or more devices that allow one or more users C to enter information into, and/or receive information from, system 10. In some embodiments, algorithm 630 uses the user C entered information to identify an FP 70 in response to an FPR 170 (e.g. FP 70 is identified by algorithm 630 based on at least the user C entered information).

CDD 200 can comprise a data device as defined herein. CDD 200 comprises user interface 210, which can include various user input components 211 and/or user output components 212, also as defined herein.

CDD 200 can comprise one or more functional elements, such as functional element 299 shown and described herein.

In some embodiments, system 10 includes SDD 300, which can comprise one or more devices that allow one or more users S to enter information into, and/or receive information from, system 10. In some embodiments, algorithm 630 uses the user S entered information to identify an FP 70 in response to an FPR 170 (e.g. FP 70 is identified by algorithm 630 based on at least the user S entered information).

SDD 300 can comprise a data device as defined herein. SDD 300 comprises user interface 310, which can include various user input components 311 and/or user output components 312, also as defined herein.

SDD 300 can comprise one or more functional elements, such as functional element 399 shown and described herein.

In some embodiments, system 10 includes SMDD 400, which can comprise one or more devices that allow one or more users M to enter information into, and/or receive information from, system 10. In some embodiments, algorithm 630 uses the user M entered information to identify an FP 70 in response to an FPR 170 (e.g. FP 70 is identified by algorithm 630 based on at least the user M entered information).

SMDD 400 can comprise a data device as defined herein. SMDD 400 comprises user interface 410, which can include various user input components 411 and/or user output components 412, also as defined herein.

SMDD 400 can comprise one or more functional elements, such as functional element 499 shown and described herein.

In some embodiments, system 10 includes PDxD 800, which can comprise one or more diagnostic devices that interface with user P to obtain diagnostic information related to user P. PDxD 800 can comprise a device that is implanted within user P, placed on the skin of user P, and/or maintained in close proximity to user P.

PDxD 800 can comprise a user interface, which can include various user input components and/or user output components, as defined herein.

PDxD 800 can comprise one or more functional elements, such as functional element 899 as described herein.

PDxD 800 can be configured to obtain diagnostic information selected from the group of: activity level information (e.g. as measured by a patient-worn activity tracking device); motion information; blood glucose information; blood pressure information; heart rate information; respiration information; pH information; digestive information; sleep information; sleep apnea information; and combinations of one, two, or more of these.

PDxD 800 can be configured to determine whether one or more FPs 70 were ingested by user P. For example, PDxD 800 can comprise a blood glucose monitor that confirms the caloric intake of one or more FPs 70 were ingested by user P. In some embodiments, PDxD 800 comprises a diagnostic device configured to provide information (e.g. patient physiologic information) selected from the group consisting of: blood glucose; blood gas such as blood oxygen (e.g. via a pulse oximeter); blood pressure; heart rate; patient activity; respiration; perspiration; breath content (e.g. breath alcohol content and/or other content of the patient's breath); patient position (e.g. lying down, sitting, or standing); and combinations thereof.

In some embodiments, system 10 includes PTxD 900, which can comprise one or more therapeutic devices that interface with user P such as to provide one or more therapies to user P.

PTxD 900 can comprise a user interface, which can include various user input components and/or user output components, as defined herein.

PTxD 900 can comprise one or more functional elements, such as functional element 999 as described herein.

PTxD 900 can be configured to provide a therapy, such as when PTxD 900 comprises a therapy-providing device selected from the group consisting of: drug or other agent delivery device such as an insulin delivery device or an oxygen providing device; external device; implanted device; pacemaker; defibrillator; drug (itself); pain control device; stimulator (e.g. implanted or external stimulator); respiration device; guided meditation device; ambulation assist device; sleep apnea device; and combinations of one, two, or more of these. In some embodiments, algorithm 630 is configured to analyze therapeutic information provided by PTxD 900 with information related to FP 70 provided by system 10 (e.g. FP 70 ingested by the patient), such as to provide feedback information (e.g. to a clinician of user P) regarding the impact of FP 70 on a therapy provided by PTxD 900, and vice versa.

PTxD 900 can comprise a device configured to prepare and/or dispense an FP 70. For example, PTxD 900 can be configured to deliver (e.g. automatically deliver) an FP 70 comprising one or more medicinal drugs, vitamins, minerals, supplements, and/or other substances (e.g. pills) to be taken by user P based on a condition (e.g. a medical condition) of user P. PTxD 900 can be configured to deliver an FP 70 that is configured to provide a neutralizing effect to one or more food products ingested by user P, such as when PTxD 900 delivers a neutralizing agent configured to provide chelation therapy, such as after user P has ingested fish or other food suspected of containing lead, mercury, iron, and/or arsenic. In these embodiments, the type of neutralizing agent, and/or the amount of the neutralizing agent, can be identified by algorithm 630, based on FP 70 ingested or to be ingested by user P (e.g. based on an actual or estimated quantity ingested).

In some embodiments, PTxD 900 is configured to deliver a substance (e.g. a medicinal substance, nutritional substance, or the like) based on a signal (e.g. a wireless signal) received from PU 600, such as a wireless signal that is sent based on an analysis performed by algorithm 630.

As described herein, system 10 includes one or more processing units, PU 600. PU 600 includes various electronic and/or other componentry that can be used to receive, store, analyze, and/or otherwise process data. PU 600 can include memory 620 including various memory storage components, such as volatile and/or non-volatile memory storage components. PU 600 (e.g. using memory 620) can store one or more: databases of data; tables of data (e.g. lookup tables of data); and the like. PU 600 can include one or more algorithms 630 which can analyze data and produce data representing the results of the analysis. The results of the analysis can be provided to PDD 100, such as when the analysis provides one or more options of FP 70 to be selected by a user P.

All or at least a portion of PU 600 can reside in PDD 100.

As described herein, PU 600 comprises stored data 650.

Stored data 650 can include a correlation of one or more food products with relatively undesirable health scores to a corresponding set of FPs 70 with a desirable (or at least a more desirable) health score. For example, stored data 650 can include a healthier alternative of a food product provided by a fast food restaurant.

Stored data 650 can include a correlation of one or more food products comprising a medicinal drug to a non-drug alternative which has been determined to provide (or at least is believed to provide) similar therapeutic action (e.g. turmeric as an alternative to an anti-inflammatory drug).

Stored data 650 can include food product information, such as a library of food product information used by algorithm 630 to identify one or more FPs 70 in response to user P making a food product request, FPR 170.

As described herein, PU 600 can receive, store, analyze, and/or otherwise process user P information, patient data 651. In some embodiments, one or more FPs 70 are provided by system 10 based on all, or at least a subset, of patient data 651. For example, algorithm 630 can analyze various data, including all, or at least a subset, of patient data 651, in order to identify one or more FPs 70 to be suggested (e.g. one or more FPs 70 options provided in an options list as identified by algorithm 630) for selection. The suggested FP 70 can be listed via user interface 110 of PDD 100 (e.g. in an options list), after which user P selects a desired FP 70, and system 10 provides FP 70 to user P.

Patient data 651 can comprise a database of data that includes or is otherwise based on: user P provided data 150 (e.g. patient-related data provided by user P via PDD 100), clinician provided data 250 (e.g. patient-related data provided by user C via CDD 200), system manufacturer provided data 450 (e.g. patient-related data provided by user M via SMDD 400), supplier provided data 350 (e.g. patient-related data that is at least based on data provided by a user S), and/or other information. In some embodiments, patient data 651 comprises data provided (e.g. uploaded to system 10) by a test (e.g. a diagnostic or other test performed on and/or by user P).

Patient data 651 can include information representing an extended period of time, such as at least 1 month, at least 6 months, at least 1 year, at least 3 years, and/or at least 5 years. In some embodiments, patient data 651 includes information representing a period of time representing the majority of user P's lifetime, such as when user P is afflicted with a chronic and/or otherwise severe medical condition. In some embodiments, algorithm 630 identifies one or more FPs 70 (e.g. one or more FPs 70 to be suggested to user P) based on the patient data 651 representing this extended period of time.

Patient data 651 can include information related to the past, present, and/or future (e.g. and can include information related to that timing), such as past, present, and/or future user P location information. For example, algorithm 630 can identify an FP 70 that is available at a store, restaurant, and/or other FP 70 provider that is in relatively close proximity to the user P (e.g. proximate the current location of user P or a location in which user P intends to ingest the FP 70). In some embodiments, user P provides to system 10 patient data 651 that includes “proximity requirement information”, such as information to be used to identify a supplier of an FP 70 that is “within X miles”, “within a X minute drive” (e.g. including traffic considerations), and/or “within an X minute walk”. In these embodiments, algorithm 630 can be configured to identify one or more FPs 70 based on the user P provided proximity requirement information.

Patient data 651 can include data provided by PDxD 800 and/or PTxD 900.

Patient data 651 can include information related to various parameters of user P, such as a parameter selected from the group consisting of: sex; race; age; height; weight; body mass index (BMI); presence of one or more medical conditions; patient health information (e.g. as described herein); recent patient information; and combinations of one, two, or more of these.

Patient data 651 can include data provided by a sensor of system 10, such as a sensor-based functional element 199, such as to provide information selected from the group consisting of: user P location information (e.g. as provided by a GPS sensor of system 10); user P physiologic information (e.g. as provided by an implanted or other physiologic sensor of system 10); and combinations of these. In some embodiments, algorithm 630 identifies an FP 70 based on user P location information (e.g. as provided by a GPS sensor of system 10), such as to identify a local store, restaurant, and/or other FP 70 provider that can provide the FP 70.

Patient data 651 can include patient health information, such as data received from user P, user C, and/or another source. For example, patient data 651 can include patient health information selected from the group consisting of: medical condition information (e.g. known or suspected presence of one or more medical conditions such as heart disease, neurological disease, Alzheimer's disease, Crohn's disease, celiac disease, diabetes, fatty liver, polycystic ovarian syndrome, and/or other diseases or disorders); blood information such as blood component level information; cholesterol information; testosterone information; estrogen level; biomarker level; urine information; biopsy information; histology information; blood flow information such as restricted artery information; bone information such as osteoporosis information; genetic information; genetic predisposition information; vitamin and/or mineral level information; sleep apnea information; allergy information; and combinations of one, two, or more of these. In some embodiments, patient health information comprises genetic and/or other data received from a DNA analysis company, such as information that is uploaded into PU 600 via the internet or otherwise. In some embodiments, algorithm 630 identifies one or more FPs 70 (e.g. one or more FPs 70 to be suggested to user P) based on the patient health information, such as when algorithm 630 identifies an FP 70 to treat (e.g. improve the condition of) or at least not adversely affect a medical condition of user P, and/or when algorithm 630 identifies a food to be avoided (e.g. due to an allergy or medical condition of user P) from being included in an FP 70. Patient health information that is included in patient data 651 can comprise information selected from the group consisting of: data collected in a patient physical examination (e.g. an annual physical exam performed by the patient's primary care physician or otherwise); data collected in a patient physiologic test such as a blood test; data collected in a patient imaging procedure (e.g. an imaging procedure producing one or more: X-rays, magnetic resonance images, PET scans, CT scans, and the like); data collected in a clinician visit (e.g. a visit performed to treat a temporary or chronic medical condition of the patient); and combinations of these. In some embodiments, algorithm 630 is adjusted on a temporal basis (e.g. adjusted routinely within a maximum time period), such as to ensure inclusion of recent patient health information in one or more analyses performed to identify FP 70. For example, an adjustment of algorithm 630 can be performed at least once per year, at least once every 6 months, and/or at least once every 3 months. In some embodiments, system 10 is configured to prevent the identification of FP 70 (e.g. by algorithm 630), if patient health information is not updated or at least confirmed for accuracy (“updated” herein) at least once per year, at least once every 6 months, or at least once every 3 months. For example, system 10 can be configured to enter a “locked”, or “out of date” mode if at least a portion of patient data 651 (e.g. at least a portion of patient clinical information of patient data 651) is not updated within a maximum time period.

Patient data 651 can include patient preference information, such as preference data received from user P, such as data selected from the group consisting of: patient likes and/or dislikes (e.g. food product likes and/or dislikes of user P); FP 70 ingestion location preference; FP 70 pickup location preference; FP 70 delivery time and/or date preference; user P patient goal information (e.g. as described herein); and combinations of one, two, or more of these. In some embodiments, algorithm 630 identifies one or more FPs 70 (e.g. one or more FPs 70 to be suggested to user P) based on the patient preference information, such as when algorithm 630 identifies an FP 70 that includes ingredients that user P likes to ingest and/or is convenient for user P to acquire and/or ingest.

Patient data 651 can include patient location information, such as patient location that is provided (manually) by user P, or information that is provided by a sensor of system 10 (e.g. a GPS or other location-providing sensor such as functional element 199). In some embodiments, algorithm 630 identifies one or more FPs 70 (e.g. one or more FPs 70 to be suggested to user P) based on the patient location information, such as when algorithm 630 identifies an FP 70 to be provided by a supplier in the relative vicinity of the user P (e.g. proximate a current or future location of user P).

Patient data 651 can include patient pantry information, such as information related to ingredients, food, and/or other FP 70 that is currently present at the user P location (e.g. within the pantry or other food storage location in the user P's home, office, or other convenient location). In some embodiments, algorithm 630 identifies one or more FPs 70 (e.g. one or more FPs 70 to be suggested to user P) based on the patient pantry information (e.g. when patient data 651 currently includes patient location information representing user P being home, and/or user P simply indicates their present location to be at home), such as when algorithm 630 identifies an FP 70 to be prepared by user P based on the patient pantry information.

Patient data 651 can include patient goal information, such as information related to: a weight-loss goal; a disease-prevention goal; a personal health goal; an activity goal (e.g. ability to run a particular length race); and combinations of one, two, or more of these. In some embodiments, algorithm 630 identifies one or more FPs 70 (e.g. one or more FPs 70 to be suggested to user P) based on the patient goal information, such as when algorithm 630 identifies an FP 70 that tends to cause the user P to achieve a goal (e.g. an algorithm that is biased towards successful completion of the goal).

Patient data 651 can include patient appetite level information, such as information related to user P's current desire for a particular quantity of food to be ingested (e.g. slightly hungry versus very hungry). In some embodiments, algorithm 630 identifies one or more FPs 70 (e.g. one or more FPs 70 to be suggested to user P) based on the patient appetite level information, such as when algorithm 630 identifies a suggested quantity of an FP 70 that correlates with the appetite level of user P (e.g. algorithm 630 is biased toward identifying FP 70 in order to achieve satiety of the patient's hunger level without over eating).

Patient data 651 can include patient fear information, such as information related to user P's current desire to avoid a particular medical condition, such as cancer, heart disease, and/or Alzheimer's disease. In some embodiments, algorithm 630 identifies one or more FPs 70 (e.g. one or more FPs 70 to be suggested to user P) based on the patient fear information, such as when algorithm 630 identifies an FP 70 known or otherwise believed to potentially reduce the risk of contracting the particular medical condition (e.g. algorithm 630 is biased towards identifying FP 70 that is known or suspected to treat and/or reduce the likelihood of one or more medical conditions that the user P desires to avoid).

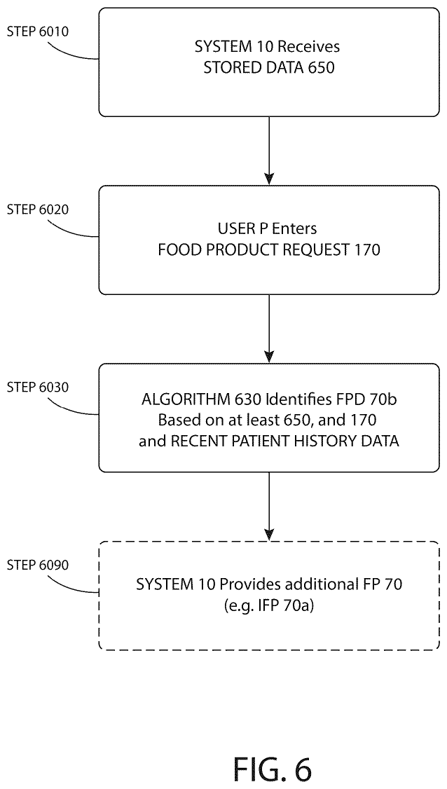

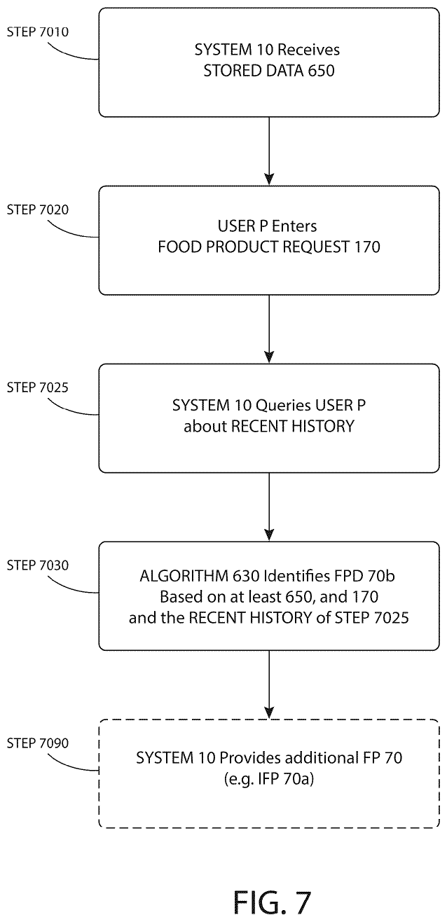

Patient data 651 can include recent patient history data, such as information related to user P's recent history, as described herein. In some embodiments, algorithm 630 identifies one or more FPs 70 (e.g. one or more FPs 70 to be suggested to user P) based on the recent patient history data, such as when algorithm 630 identifies an FP 70 based on recent activity of user P (e.g. recent food ingestion, recent exercise or other physical activity, recent internet activity, and/or recent location), such as is described herein in reference to FIGS. 6, 7, and/or 8. In some embodiments, recent patient history data includes a qualitative and/or quantitative user P-provided assessment of current health status of user P. For example, user P can provide information related to being tired, sluggish, and the like, after which algorithm 630 identifies an FP 70 to address (e.g. improve upon) the user P-provided assessment.

Patient data 651 can include patient medication information, such as information related to one or more medicinal drugs taken by user P (e.g. recently or otherwise). In some embodiments, algorithm 630 identifies or more FPs 70 (e.g. one or more FPs 70 to be suggested to user P) based on the patient medication information, such as when algorithm 630 identifies an FPD 70b to avoid a food product that should not be ingested with the drug and/or to ingest a food product known or otherwise believed to enhance the efficacy of the drug.

Patient data 651 can include patient clinical procedure information, such as information related to one or more surgeries, endoscopies, angioplasties, and/or other clinical procedures to be performed and/or previously performed upon user P (e.g. soon, recently or otherwise). In some embodiments, algorithm 630 identifies one or more FPs 70 (e.g. one or more FPs 70 to be suggested to user P) based on the patient clinical procedure information, such as when algorithm 630 identifies an FP 70 to avoid a food product that could conflict with the clinical procedure, and/or to ingest a food product known or otherwise believed to enhance the clinical procedure.

As described herein, PU 600 can receive, store, analyze, and/or otherwise process generic clinical data 652. In some embodiments, one or more FPs 70 are provided by system 10 based on all, or at least a subset, of generic clinical data 652. For example, algorithm 630 can analyze various data, including all, or at least a subset, of generic clinical data 652, in order to identify one or more FPs 70 to be suggested (e.g. one or more FPs 70 options provided in an options list) for selection. In some embodiments, algorithm 630 can analyze various data, including all, or at least a subset, of generic clinical data 652, as well as all, or at least a subset, of patient clinical data 651, in order to identify one or more FPs 70 to be suggested (e.g. one or more FPs 70 options provided in an options list) for selection. The suggested FP 70 can be listed via user interface 110 of PDD 100, after which user P selects a desired FP 70, and system 10 provides FP 70 to user P.

Generic clinical data 652 can comprise a database of data that includes or is otherwise based on: clinician provided data 250 (e.g. generic clinical data provided by user C via CDD 200); supplier provided data 350 (e.g. generic clinical data provided by a user S via SDD 300); system manufacturer provided data 450 (e.g. generic clinical data provided by a user M via SMDD 400); and/or other information.

Generic clinical data 652 can be shared among multiple PDDs 100, such as to share the data among multiple user Ps each having at least one PDD 100.

Generic clinical data 652 can include information about the relationship between a food product and a medical condition, and/or other clinical information related to a food product. Typical generic clinical data can include information such as: spinach may have a positive impact on Alzheimer's disease; turmeric may have a positive impact on joint pain and other inflammatory conditions; peppermint may treat an upset stomach; and the like.

Generic clinical data 652 can provide information for certain food products to tend to be avoided from inclusion in FP 70, such as soy, wheat grass, and/or goji berries (e.g. when certain user P conditions are present in which avoiding ingestion of one or more of those products should be considered); and/or data 652 can provide information for certain food products to tend to be included in FP 70, such as polyphenol-rich foods, aronia berries, pomegranates, mulberries, blueberries, cranberries, and/or blackberries (e.g. where certain user P conditions are present in which ingestion of one or more of those products can provide a benefit).

Generic clinical data 652 can include diet information, such as food products to be included and/or avoided to achieve a ketogenic diet, a low carbohydrate diet, and the like.

Generic clinical data 652 can include clinical information from various human subjects separate from user P, such as when system 10 characterizes user P in one or more ways (e.g. sex, age, weight, height, body surface area, race, and the like), and algorithm 630 utilizes generic clinical data 652 from various other human subjects in similar categories to user P to recommend (e.g. identify) an FP 70 for ingestion.

As described herein, PU 600 can receive, store, analyze, and/or otherwise process generic supplier data 653. Supplier data 653 can include food product information (e.g. information for food products offered by the supplier), such as a library of food product information used by algorithm 630 to identify one or more FPs 70 in response to user P making a food product request, FPR 170.

In some embodiments, one or more FPs 70 are provided by system 10 based on all, or at least a subset, of supplier data 653. For example, algorithm 630 can analyze various data, including all, or at least a subset, of supplier data 653, in order to identify one or more FPs 70 to be suggested (e.g. one or more FPs 70 options provided in an options list) for selection. In some embodiments, algorithm 630 can analyze various data, including all, or at least a subset, of supplier data 653 as well as all, or at least a subset, of patient clinical data 651 and/or all, or at least a subset, of generic clinical data 652, in order to identify one or more FPs 70 to be suggested (e.g. one or more FPs 70 options provided in an options list) for selection. The suggested FP 70 can be listed via user interface 110 of PDD 100, after which user P selects a desired FP 70, and system 10 provides FP 70 to user P.

Supplier data 653 can comprise a database of data that includes or is otherwise based on: clinician provided data 250 (e.g. generic clinical data provided by user C via CDD 200); supplier provided data 350 (e.g. generic clinical data provided by a user S via SDD 300); system manufacturer provided data 450 (e.g. generic clinical data provided by a user M via SMDD 400); and/or other information.

Supplier data 653 can be shared among multiple PDDs 100, such as to share the data among multiple user Ps each having at least one PDD 100.

Supplier data 653 can comprise one or more food product parameters, as described herein.

Supplier data 653 can comprise location information, such as location information related to one or more restaurants, grocery stores, and/or other suppliers that provide FP 70.

Supplier data 653 can include tables of FPs 70 as provided by different suppliers, as well as information related to those food products, such as information selected from the group consisting of: price information; lead time information; availability information, such as availability by location; ingredient information (e.g. ingredients of a multi-ingredient food product); health information, such as health score information; manufacturing location (e.g. farm location and/or other manufacturing location of the FP 70); and combinations of one, two, or more of these.

Supplier data 653 can include information related to one more restaurant-based suppliers, such as information related to a menu, each food product available on that menu (e.g. including ingredients), and the address (physical location) of the restaurant.

In some embodiments, supplier data 653 comprises information related to one or more FPs 70 provided by one or more users S, as well as “correlating information” provided by one or more users C and/or one or more users M. For example, a clinician-based user C or user M can provide a health score or other clinician-provided information (correlating information) for one or more FPs 70 provided by a restaurant, grocery store, or other food product provider. Algorithm 630 can be configured to utilize the clinician-provided correlating information to identify one or more FPs 70 to recommend to user P.

As described herein, PU 600 can receive, store, analyze, and/or otherwise process other data 654. In some embodiments, one or more FPs 70 are provided by system 10 based on all, or at least a subset, of other data 654. For example, algorithm 630 can analyze various data, including all, or at least a subset, of other data 654, in order to identify one or more FPs 70 to be suggested (e.g. one or more FPs 70 options provided in an options list) for selection. In some embodiments, algorithm 630 can analyze various data, including all, or at least a subset, of other data 654, as well as all, or at least a subset, of patient clinical data 651, all, or at least a subset, of generic clinical data 652, and/or all, or at least a subset, of supplier data 653, in order to identify one or more FPs 70 to be suggested (e.g. one or more FPs 70 options provided in an options list) for selection. The suggested FP 70 can be listed via user interface 110 of PDD 100, after which user P selects a desired FP 70, and system 10 provides FP 70 to user P.

Other data 654 can include food product information, such as when other data 654 includes a library of food product information used by algorithm 630 to identify one or more FPs 70 in response to user P making a food product request, FPR 170.

Other data 654 can comprise a database of data that includes or is otherwise based on: user provided data 150 (e.g. data provided by user P or other user via PDD 100); clinician provided data 250 (e.g. generic clinical data provided by user C via CDD 200); supplier provided data 350 (e.g. generic clinical data provided by a user S via SDD 300); system manufacturer provided data 450 (e.g. generic clinical data provided by a user M via SMDD 400); and/or other information.

Other data 654 can be shared among multiple PDDs 100, such as to share the data among multiple user Ps each having at least one PDD 100.

Other data 654 can comprise data related to transportation between user P and a current location of FP 70, such as map data (e.g. street map data) and/or traffic data. In some embodiments, algorithm 630 identifies one or more FPs 70 (e.g. one or more FPs 70 to be suggested to user P) based on the map data and/or traffic data, such as when algorithm 630 identifies an FP 70 based on the amount of time for FP 70 to be delivered to user P and/or for user P to travel to FP 70 (e.g. to travel to a restaurant or other food product provider). For example, user P can select an FP 70 based on this amount of time (e.g. select one FP 70 over another to reduce this amount of time).

As described herein, PU 600 can include one or more algorithms, algorithm 630. In some embodiments, algorithm 630 can be configured to analyze various data, including a food product request, FPR 170, and other stored data 650, in order to identify one or more FPs 70 to be suggested (e.g. one or more FP 70 options provided in an options list) for selection. The suggested FP 70 can be listed via user interface 110 of PDD 100, after which user P selects a desired FP 70, and system 10 provides FP 70 to user P.

In some embodiments, algorithm 630 comprises confirmation routine, as described herein, such as a routine in which a user U (e.g. a user C and/or user P) approves an addition, deletion, and/or change to system 10, such as an addition, deletion and/or change to algorithm 630, to stored data 650, and/or to other data or formula of system 10. For example, approval via a confirmation routine of algorithm 630 can be required (e.g. by a clinician or guardian of user P) in order to change a stored value related to a parameter selected from the group consisting of: a food product; a rating to a food product, such as a health score; patient data, such as patient allergy data; a risk assessment, such as a risk associated with a particular food product for user P; and combinations of one, two, or more of these.

In some embodiments, algorithm 630 comprises an algorithm configured to estimate food products ingested by user P (e.g. estimate the specific food products ingested and/or the quantity of those food products ingested). For example, the algorithm 630 can include a bias that assumes an FP 70 selected by user P is actually ingested by user P. In some embodiments, the algorithm 630 is configured to request confirmation from user P of FP 70 ingestion. In some embodiments, the algorithm 630 utilizes information received from a sensor (e.g. a microphone, a camera, and/or other sensor) and/or from a diagnostic device (e.g. a blood glucose meter or other diagnostic device) of system 10, in order to estimate ingestion of food products ingested.

System 10 can include one or more functional elements, such as functional elements 199, 299, 399, 499, 899, and/or 999 shown in FIG. 1. Each functional element 199, 299, 399, 499, 899, and/or 999 can comprise one or more sensors, transducers, and/or other functional elements.

In some embodiments, functional elements 199, 299, 399, 499, 899, and/or 999 comprise one or more sensors configured to record information of user P, such as information selected from the group consisting of: activity information (e.g. an accelerometer or other motion sensor, such as a sensor that can provide information related to calories burned by user P); physiologic information (e.g. a blood glucose sensor, a respiration sensor, an electrode or other electrical sensor, and the like); location information (e.g. a GPS sensor used to determine user P location); posture information (e.g. information related to user P being in a lying down, sitting, or standing position); and combinations of one, two, or more of these.

In some embodiments, functional elements 199, 299, 399, 499, 899, and/or 999 comprise one or more transducers. For example, functional element 199 of PDD 100 can comprise a sound (e.g. speaker), visible (e.g. light), and/or vibrational sensor, each of which can be configured to alert user P (e.g. to alert user P of an upcoming event), such as: a time to ingest FP 70, such as a time to take a FP 70 comprising a drug, vitamin, mineral, supplement, and/or other medication; a time to exercise; and/or a time to initiate travel to a restaurant or other supplier of FP 70.

In some embodiments, functional elements 199, 299, 399, 499, 899, and/or 999 comprise an observational device. For example, functional element 199 of PDD 100 can comprise a camera and/or microphone, such as to record a user P command, request, feedback, or other user P-provided information. For example, a functional element configured as an observational device can record a user P like or dislike of a food product, can record a question of user P, can confirm an FP 70 was ingested by user P, and the like. In some embodiments, a functional element 199, 299, 399, 499, 899, and/or 999 comprises an observational device configured to provide information used by algorithm 630 to estimate ingestion of food products by user P.

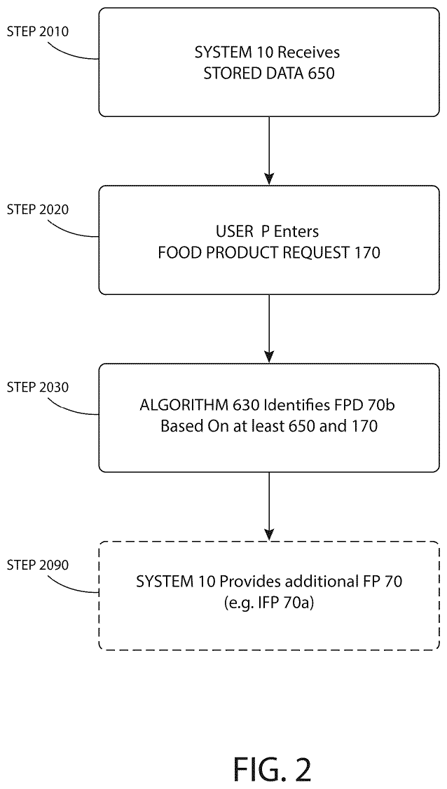

FIGS. 2 through 15 described herein are flow charts of various uses of system 10 and are each described in reference to the components of system 10 described herein in reference to FIG. 1.

Referring now to FIG. 2, a flow chart of a method for a patient to obtain a food product is illustrated, consistent with the present inventive concepts. In Step 2010, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years.

In Step 2020, user P enters a food product request, FPR 170, such as by entering data into user interface 110 of PDD 100.

In Step 2030, algorithm 630 analyzes the FPR 170 and the stored data 650, and identifies FP 70 based on the analysis. System 10 can provide the identified FP 70 to user P, where the FP 70 provided can comprise FPD 70b. FPD 70b can include a description of one or more FPs 70 to be ingested by user P. FPD 70b can be provided to user P via user interface 110 of PDD 100 or via any display or printed matter. In some embodiments, system 10 first provides a list of one or more suggested FPs 70 identified by algorithm 630, and user P selects all, or at least a subset, of the suggested FPs 70. Subsequently, system 10 can provide FPD 70b to user P based on the selection.

An optional Step 2090 can be performed, in which system 10 further provides additional FP 70 to user P, which can include additional FPD 70b (e.g. in addition to what was provided in Step 2030), and/or include actual food product to be ingested, IFP 70a. IFP 70a can be provided by a delivery service, or by a supplier of IFP 70a (e.g. an internet-based food provider that ships food product via conventional means, a restaurant, a meal kit recipe delivery service, a mobile food-ordering company, or a grocery store).

System 10 can be configured to allow a user U (e.g. user P) to go back one or more steps, and/or advance one or more steps. For example, user P may be dissatisfied with the FPD 70b identified by algorithm 630 in Step 2030, and subsequently return to perform Step 2020 at least a second time (e.g. to modify FPR 170).

System 10 can be configured to require one or more additions, deletions, and/or other changes to a system 10 parameter (e.g. one or more changes to algorithm 630 and/or stored data 650) to be approved or otherwise “confirmed” by one or more users U of system 10, such as a confirmation by a clinician-based user C, a supplier-based user S, a manufacturer-based user M, and/or by user P. For example, system 10 can include a “confirmation routine” that is performed to change certain parameters, such as when a clinician of user P is required to confirm a change to one or more of: allergy information; medical condition information; food product benefit information; food ingestion information; and the like. Without successful confirmation, system 10 can leave the parameter unchanged, and/or system 10 could de-activate the parameter (e.g. not include it in use by algorithm 630 or otherwise). In some embodiments, confirmation to change certain parameters is required by two or more of these users U (e.g. user P and another user U, user C and another user U, user S and another user U, and/or user M and another user U).

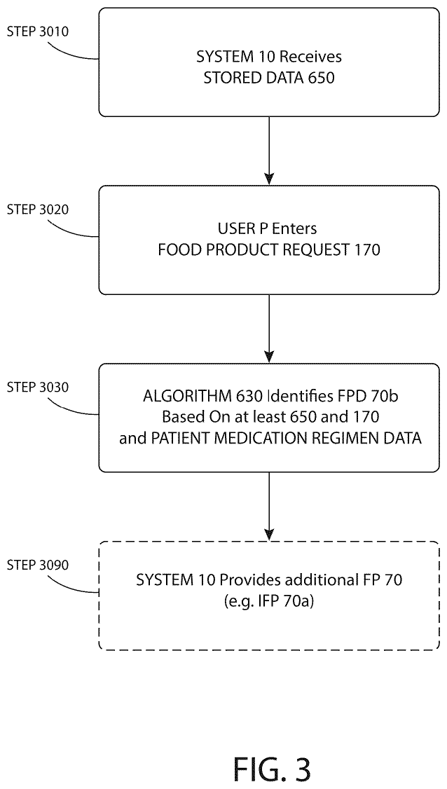

Referring now to FIG. 3, a flow chart of a method for a patient to obtain a food product based on a medication regimen of the patient is illustrated, consistent with the present inventive concepts. In Step 3010, system 10 receives various data (e.g. at least patient medication information as described herein), which is stored by PU 600 as stored data 650. Stored data 650 can also include patient provided data 150, clinician provided data 250, supplier provided data 350, system manufacturer provided data 450, diagnostic device-provided data 850, therapeutic device-provided data 950, and/or other data, which is also stored by PU 600 as stored data 650. Stored data 650 can comprise data collected over minutes, hours, months, and/or years, such as patient medication information which covers time periods of minutes, hours, months, and/or years (e.g. a library of medications and times of ingestion for user P that spans minutes, hours, months, and/or years).

In Step 3020, user P enters a food product request, FPR 170, such as by entering data into user interface 110 of PDD 100, such as is described herein in reference to Step 2020 of FIG. 2.

In Step 3030, algorithm 630 analyzes the FPR 170 and the stored data 650, and identifies FP 70 based on the analysis. System 10 can provide to user P one or more FPs 70 or suggestions for FPs 70 based on the analysis by algorithm 630, such as is described herein in reference to Step 2030 of FIG. 2. In the embodiment of FIG. 3, the analysis performed by algorithm 630 is based on patient medication information (e.g. patient data 651 comprising at least patient medication information). For example, FP 70 can be identified by algorithm 630 to avoid ingredients known or suspected to be in conflict with a particular pharmaceutical drug or other health agent that user P has taken in the past (e.g. within a month, or a week) or that user P is currently taking.

An optional Step 3090 can be performed, in which system 10 further provides additional FP 70, which can include additional FPD 70b (e.g. in addition to what was provided in Step 3030) and/or actual food product to be ingested, IFP 70a, such as is described herein in reference to Step 2090 of FIG. 2.

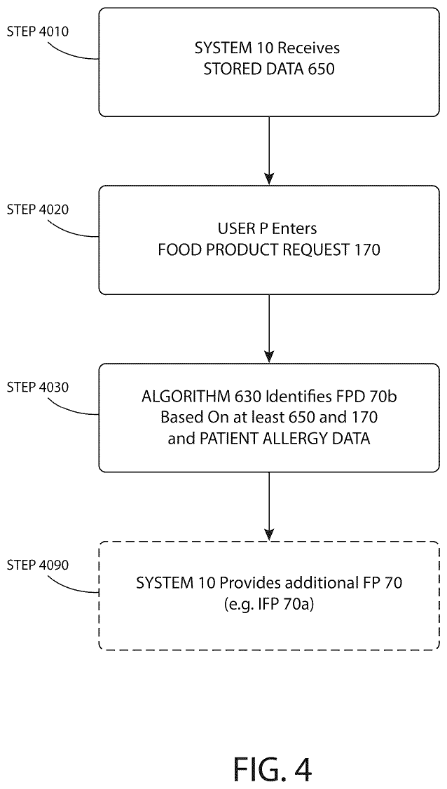

Referring now to FIG. 4, a flow chart of a method for a patient to obtain a food product based on an allergy of the patient is illustrated, consistent with the present inventive concepts. In Step 4010, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years. In the embodiment of FIG. 4, stored data 650 comprises at least patient data 651 that includes information related to one or more user P allergies, “patient allergy data” herein. As described herein, patient allergy data can comprise allergy data, food sensitivity data, food intolerance data, and/or data related to any food that results in an adverse reaction to user P (e.g. an adverse reaction that occurs when user P ingests the food or simply is in close proximity to the food). Patient allergy data can include data related to a quantity, such as a minimum quantity, of a food product that would result in an adverse reaction, “allergic threshold data”herein.

In Step 4020, user P enters a food product request, FPR 170, such as by entering data into user interface 110 of PDD 100, such as is described herein in reference to Step 2020 of FIG. 2.

In Step 4030, algorithm 630 analyzes the FPR 170 and the stored data 650, and identifies FP 70 based on the analysis. System 10 can provide to user P one or more FPs 70 or suggestions for FPs 70 based on the analysis by algorithm 630, such as is described herein in reference to Step 2030 of FIG. 2. In the embodiment of FIG. 4, the analysis performed by algorithm 630 is based on at least patient allergy data of patient data 651. For example, FP 70 can be identified by algorithm 630 to avoid an adverse reaction to a food product to which user P is allergic. In some embodiments, algorithm 630 performs an analysis based on both patient medication information (e.g. as described herein in reference to FIG. 3) and patient allergy data. In some embodiments, algorithm 630 identifies FP 70 based on allergic threshold data. In some embodiments, algorithm 630 performs an analysis based on recently ingested food products (e.g. known or estimated by system 10), such as to compare the levels of a particular food product ingested by user P, to user P's allergic threshold data (e.g. when algorithm 630 avoids identifying an FP 70 only when future ingestions of that FP 70 would exceed the particular threshold).

An optional Step 4090 can be performed, in which system 10 further provides additional FP 70, which can include additional FPD 70b (e.g. in addition to what was provided in Step 4030) and/or actual food product to be ingested, IFP 70a, such as is described herein in reference to Step 2090 of FIG. 2.

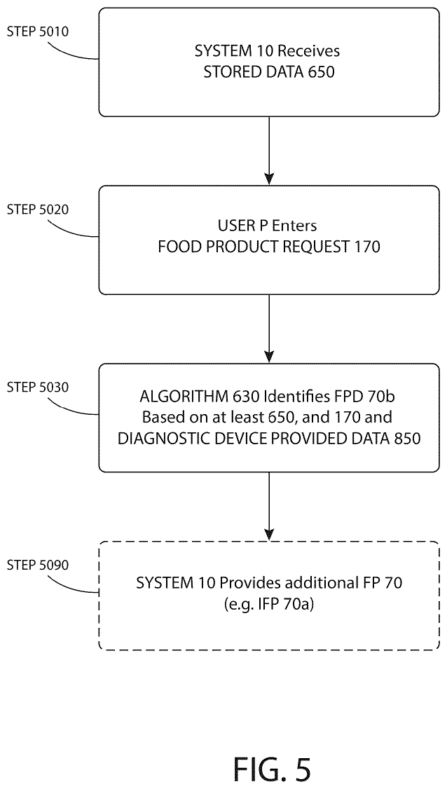

Referring now to FIG. 5, a flow chart of a method for a patient to obtain a food product based on sensor data is illustrated, consistent with the present inventive concepts. In Step 5010, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years. In the embodiment of FIG. 5, stored data 650 comprises at least data provided by PDxD 800, diagnostic device-provided data 850. For example, PDxD 800 can provide data related to one or more user P physiologic parameters, such as activity level, blood glucose level, blood oxygen level, heart rate, blood pressure, respiration, and the like.

In Step 5020, user P enters a food product request, FPR 170, such as by entering data into user interface 110 of PDD 100, such as is described herein in reference to Step 2020 of FIG. 2.

In Step 5030, algorithm 630 analyzes the FPR 170 and the stored data 650, and identifies FP 70 based on the analysis. System 10 can provide to user P one or more FPs 70 or suggestions for FPs 70 based on the analysis by algorithm 630, such as is described herein in reference to Step 2030 of FIG. 2. In the embodiment of FIG. 5, the analysis performed by algorithm 630 is based on diagnostic device-provided data 850. For example, FP 70 can be identified by algorithm 630 to improve an undesired health state indicated by data 850, and/or to maintain a desired health state indicated by data 850.

In some embodiments, algorithm 630 performs an analysis based on two, or all of: patient medication information (e.g. as described herein in reference to FIG. 3); patient allergy data (e.g. as described herein in reference to FIG. 4); and/or patient diagnostic device data 850.

An optional Step 5090 can be performed, in which system 10 further provides additional FP 70, which can include additional FPD 70b (e.g. in addition to what was provided in Step 5030) and/or actual food product to be ingested, IFP 70a, such as is described herein in reference to Step 2090 of FIG. 2.

In some embodiments, PDxD 800 is configured to produce diagnostic device-provided data 850 comprising non-physiologic information, such as when PDxD 800 comprises a GPS device configured to provide location information for user P. In these embodiments, algorithm 630 can identify FP 70 based on the user P location (e.g. identify IFP 70a based on a restaurant or other food supplier that is at a location in relative proximity to the user P, such as can be determined based on proximity requirement information as described herein). In these embodiments, PDxD 800 can be further configured to produce physiologic information, such as physiologic information also used by algorithm 630 to identify FP 70.

Referring now to FIG. 6, a flow chart of a method for a patient to obtain a food product based on recent patient history is illustrated, consistent with the present inventive concepts. In Step 6010, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years. In the embodiment of FIG. 6, stored data 650 comprises patient data 651 which includes at least recent patient history data, as described herein.

In Step 6020, user P enters a food product request, FPR 170, such as by entering data into user interface 110 of PDD 100, such as is described herein in reference to Step 2020 of FIG. 2.

In Step 6030, algorithm 630 analyzes the FPR 170 and the stored data 650, and identifies FP 70 based on the analysis. System 10 can provide to user P one or more FPs 70 or suggestions for FPs 70 based on the analysis by algorithm 630, such as is described herein in reference to Step 2030 of FIG. 2. In the embodiment of FIG. 6, the analysis performed by algorithm 630 is based on at least recent patient history data.

In some embodiments, FP 70 is identified by algorithm 630 based on food that was recently ingested by user P (e.g. food that is known by system 10 to have been ingested, and/or estimated by system 10 to have been ingested). In these embodiments, a quantity (e.g. a high level or low level) and/or type of food(s) of FP 70 can be identified by algorithm 630 to balance and/or otherwise be compatible with (“balance” herein) a quantity (e.g. a low level or high level, respectively) and/or type of food that was recently ingested. In some embodiments, a level of a substance (e.g. a vitamin and/or mineral) is balanced by algorithm 630, such as when algorithm 630 is biased to maintain a minimum level of a substance over a time period (e.g. a day, or a week), and/or to prevent exceeding a maximum level of a substance over a time period (e.g. a day, or a week). In some embodiments, a caloric level of food products ingested is balanced by algorithm 630, such as when algorithm 630 is biased to identify FP 70 in order to maintain a minimum caloric intake over a time period (e.g. a day, or a week), and/or to prevent exceeding a maximum level of caloric intake over a time period (e.g. a day, or a week). In some embodiments, system 10 is configured to allow user P to adjust stored data 650 related to food that is known or estimated to have been ingested by user P, such that a user U (e.g. user P or another user U) can adjust such information that is inaccurate (e.g. to adjust an output of algorithm 630 that is based on recently ingested food). For example, system 10 can be configured to provide via a user interface (e.g. provided visually via a screen of user interface 110 of PDD 100) a library of information of food ingested by user P (e.g. food ingested by user P within the last day, or last week), and system 10 can be further configured to allow a user U (e.g. user P) to adjust that library of information (e.g. adjust that portion of stored data 650). In some embodiments, user P comprises a patient under 18 years of age, and a parent or guardian user U must be involved to change the ingested food information included in stored data 650 (e.g. via a confirmation routine as described herein).

In some embodiments, FP 70 is identified by algorithm 630 based on recent patient activity (e.g. as entered by user P and/or determined by PDxD 800). In these embodiments, a quantity (e.g. a high level or a low level) of FP 70 can be identified by algorithm 630 to create a balance with recent patient activity. For example, if recent user P activity has been at a low level (e.g. time spent sitting, lying down, and/or otherwise relatively inactive), algorithm 630 can be biased to identify FP 70 with a relatively low caloric level. Conversely, if recent user P activity has been at a high level (e.g. recent time has been spent exercising, working vigorously, and/or otherwise relatively active), algorithm 630 can be biased to identify FP 70 with a relatively high caloric level.

In some embodiments, algorithm 630 performs an analysis based on two, three, or all of: patient medication information (e.g. as described herein in reference to FIG. 3); patient allergy data (e.g. as described herein in reference to FIG. 4); patient diagnostic device data 850 (as described herein in reference to FIG. 5); and/or recent patient activity (e.g. recent food ingestion and/or recent patient activity).

An optional Step 6090 can be performed, in which system 10 further provides additional FP 70, which can include additional FPD 70b (e.g. in addition to what was provided in Step 6030) and/or actual food product to be ingested, IFP 70a, such as is described herein in reference to Step 2090 of FIG. 2.

Referring now to FIG. 7, a flow chart of a method for a patient to obtain a food product based on system requested recent patient history is illustrated, consistent with the present inventive concepts.

In Step 7010, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years.

In Step 7020, user P enters a food product request, FPR 170, such as by entering data into user interface 110 of PDD 100, such as is described herein in reference to Step 2020 of FIG. 2.

In Step 7025, system 10 queries user P (e.g. via user interface 110 of PDD 100) to enter recent patient history data, such as recent food ingested by user P and/or other recent user P activity. In some embodiments, system 10 queries user P whether recently provided FP 70 was ingested (e.g. food product ingested based on system 10 provided FPD 70b and/or IFP 70a).

In Step 7030, algorithm 630 analyzes the FPR 170 and the stored data 650, and identifies FP 70 based on the analysis. System 10 can provide to user P one or more FPs 70 or suggestions for FPs 70 based on the analysis performed by algorithm 630, such as is described herein in reference to Step 2030 of FIG. 2. In the embodiment of FIG. 7, the analysis performed by algorithm 630 is based on at least recent patient history data, such as the patient history data entered in Step 7025.

In some embodiments, FP 70 is identified by algorithm 630 based on recently ingested food by user P, such as is described herein in reference to FIG. 6.

In some embodiments, FP 70 is identified by algorithm 630 based on recent user P activity, such as is described herein in reference to FIG. 6.

In some embodiments, algorithm 630 performs an analysis based on two, three, or all of patient medication information (e.g. as described herein in reference to FIG. 3); patient allergy data (e.g. as described herein in reference to FIG. 4); patient diagnostic device data 850 (e.g. as described herein in reference to FIG. 5); and/or recent user P activity (e.g. as estimated by system 10 and/or as described herein in reference to FIG. 6).

An optional Step 7090 can be performed, in which system 10 further provides additional FP 70, which can include additional FPD 70b (e.g. in addition to what was provided in Step 7030) and/or actual food product to be ingested, IFP 70a, such as is described herein in reference to Step 2090 of FIG. 2.

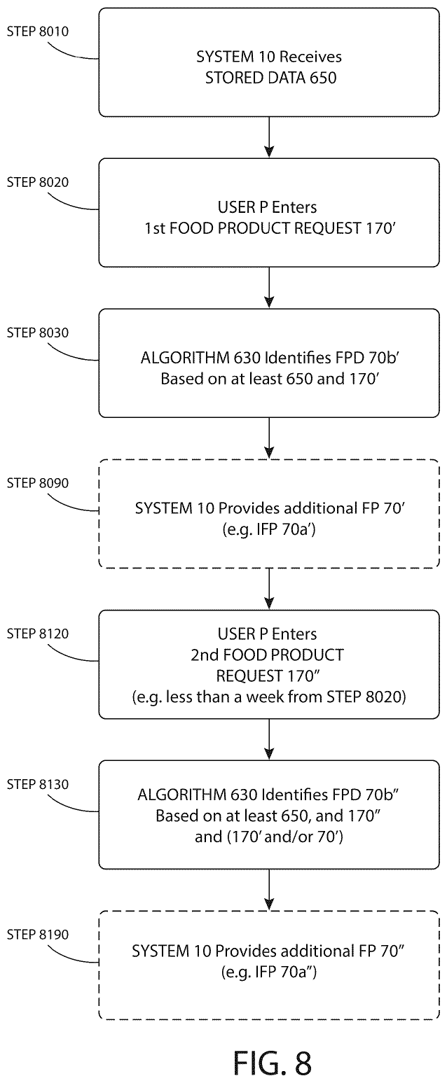

Referring now to FIG. 8, a flow chart of a method for a patient to obtain a food product based on system estimated recent patient history is illustrated, consistent with the present inventive concepts. In Step 8010, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years.

In Step 8020, user P enters a first food product request, FPR 170′, such as by entering data into user interface 110 of PDD 100, such as is described herein in reference to Step 2020 of FIG. 2.

In Step 8030, algorithm 630 analyzes the first FPR 170′and the stored data 650, and identifies FP 70 based on the analysis. System 10 can provide to user P one or more FPs 70 or suggestions for FPs 70 based on the analysis performed by algorithm 630, such as is described herein in reference to Step 2030 of FIG. 2. In the embodiment of FIG. 8, the analysis performed by algorithm 630 can be based on various data, and/or it can include a bias, each as described herein.

An optional Step 8090 can be performed, in which system 10 further provides additional food product, FP 70′, which can include additional food product data, FPD 70b′ (e.g. in addition to what was provided in Step 8030) and/or actual food product to be ingested, IFP 70a′, such as is described herein in reference to Step 2090 of FIG. 2.

In Step 8120, user P enters a second food product request, FPR 170″ (e.g. entered less than a week from performing Step 8020), such as by entering data into user interface 110 of PDD 100, such as is described herein in reference to Step 2020 of FIG. 2.

In Step 8130, algorithm 630 analyzes the second FPR 170″, stored data 650, as well as either or both of first FPR 170′and first FP 70′, and system 10 can provide to user P one or more FPs 70 or suggestions for FPs 70, such as is described herein in reference to Step 2030 of FIG. 2. The analysis performed by algorithm 630 can be biased, such as a bias that assumes that user P ingested at least a portion of first FP 70′and/or food defined by first FP 70′(i.e. defined by first FPD 70b′ of first FP 70′). In the embodiment of FIG. 8, the analysis performed by algorithm 630 can be based on various data, and/or it can include a bias, each as described herein. In some embodiments, algorithm 630 is biased to assume user P ingested an unhealthy food (e.g. more bias than assuming user P ingested a healthy food), to cause algorithm 630 to have a relatively strong bias toward healthy FPs 70. Algorithm 630 can be biased based on patient history information entered by user P and/or patient history information “estimated” by system 10, each as described herein.

In some embodiments, algorithm 630 performs an analysis based on two, three, four, or all of: patient medication information (e.g. as described herein in reference to FIG. 3); patient allergy data (e.g. as described herein in reference to FIG. 4); patient diagnostic device data 850 (e.g. as described herein in reference to FIG. 5); recent patient activity (e.g. as estimated by system 10 as described herein in reference to FIG. 6, and/or as described herein in reference to FIG. 7); and/or a previously provided FPD 70b and/or other FP 70.

An optional Step 8190 can be performed, in which system 10 further provides additional FP 70″, which can include additional FPD 70b″ (e.g. in addition to what was provided in Step 8030) and/or actual food product to be ingested, IFP 70a″, such as is described herein in reference to Step 2090 of FIG. 2.

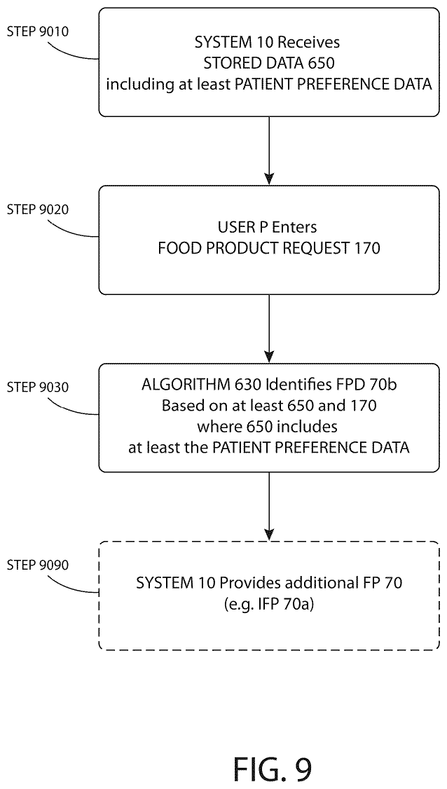

Referring now to FIG. 9, a flow chart of a method for a patient to obtain a food product based on a patient preference is illustrated, consistent with the present inventive concepts. In Step 9010, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years. In the embodiment of FIG. 9, stored data 650 comprises patient data 651 which includes at least a preference of user P (“patient preference information” as described herein), such as a food product that user P likes (e.g. desires to ingest) and/or dislikes (e.g. desires to avoid ingesting). User P preferences can be entered into system 10 via user interface 110 of PDD 100. In some embodiments, system 10 queries user P to provide preference feedback information, such as a request performed after (e.g. soon after) a particular FP 70 is provided and/or ingested. In some embodiments, the inclusion and/or avoidance preference is quantified (e.g. on a numeric scale, such as “5 stars” for foods the user P strongly favors ingesting) and/or the user P preference is qualified (e.g. via choices such as “avoid a lot”, “avoid a little”, “suggest a little”, “suggest a lot”, and the like).

In Step 9020, user P enters a food product request, FPR 170, such as by entering data into user interface 110 of PDD 100, such as is described herein in reference to Step 2020 of FIG. 2.

In Step 9030, algorithm 630 analyzes the FPR 170 and the stored data 650, including at least the patient preference data, and identifies FP 70 based on the analysis. System 10 can provide to user P one or more FPs 70 or suggestions for FPs 70 based on the analysis by algorithm 630, such as is described herein in reference to Step 2030 of FIG. 2. In the embodiment of FIG. 9, the analysis performed by algorithm 630 is biased based on one or more preferences of user P. In some embodiments, algorithm 630 provides one or more FPs 70 based on both a food to avoid, and a food to include.

In some embodiments, algorithm 630 performs an analysis based on two, three, four, five, or all of: patient medication information (e.g. as described herein in reference to FIG. 3); patient allergy data (e.g. as described herein in reference to FIG. 4); patient diagnostic device data 850 (e.g. as described herein in reference to FIG. 5); recent patient activity (e.g. as estimated by system 10 as described herein in reference to FIG. 6, and/or as described herein in reference to FIG. 7); a previously provided FPD 70b and/or other FP 70 (e.g. as described herein in reference to FIG. 8); and/or a patient preference (e.g. a patient like and/or dislike).

An optional Step 9090 can be performed, in which system 10 further provides additional FP 70, which can include additional FPD 70b (e.g. in addition to what was provided in Step 9030) and/or actual food product to be ingested, IFP 70a, such as is described herein in reference to Step 2090 of FIG. 2.

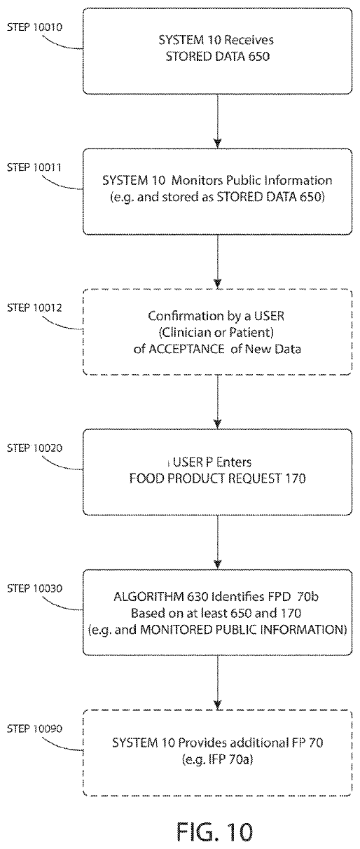

Referring now to FIG. 10, a flow chart of a method for a patient to obtain a food product based on monitored public data is illustrated, consistent with the present inventive concepts. In Step 10010, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years. In the embodiment of FIG. 10, stored data 650 comprises information received by system 10 via monitoring of public information, such as is described herein.

In Step 10011, system 10 monitors and collects (e.g. uploads) public information, such as information found on the internet, printed in journals or books, or presented at conferences. For example, one or more users M may monitor public information, and the public information can be entered into system 10 via SMDD 400. Alternatively or additionally, SMDD 400 can automatically or semi-automatically (“automatically”herein) monitor the internet and other electronic media for applicable public information.

Relevant public information includes but is not limited to: current medical practices; current nutritional practices; food product safety information (including ingredient safety information); supplier assessment information; and combinations of these. In some embodiments, the information collected is used by system 10 (e.g. by PU 600) to modify one or more algorithms of algorithm 630 (e.g. modify a bias of an algorithm, the level of a variable of an algorithm and/or used by an algorithm, and the like). In some embodiments, the information collected results in a change to stored data 650 (e.g. an addition, deletion, or modification of clinical data 652, supplier data 653, and/or other data 654).

An optional Step 10012 can be performed, in which any change made by system 10 in Step 10011 is processed via a confirmation routine (e.g. as described herein) configured to enable a user (e.g. user P, a user C, a user S, and/or a user M) to view each change (e.g. via a data device) and either allow (e.g. confirm) or prevent each change. In some embodiments, any changes (e.g. changes to algorithm 630 or stored data 650) are not implemented until proper acceptance (i.e. confirmation) via Step 10012 is performed (e.g. confirmed by a clinician and/or guardian of user P, by user P themselves, and/or by another user U of system 10). In some embodiments, multiple users U are required to confirm the change (e.g. both user P and a user C comprising a clinician of the user P).

In Step 10020, user P enters a food product request, FPR 170, such as by entering data into user interface 110 of PDD 100, such as is described herein in reference to Step 2020 of FIG. 2.

In Step 10030, algorithm 630 analyzes the FPR 170 and the stored data 650, and identifies FP 70 based on the analysis. System 10 can provide to user P one or more FPs 70 or suggestions for FPs 70 based on the analysis by algorithm 630, such as is described herein in reference to Step 2030 of FIG. 2. In the embodiment of FIG. 10, the analysis performed by algorithm 630 can be impacted by the information collected in Step 10011 (e.g. impacted by a change in algorithm 630 and/or a change in data of stored data 650).

In some embodiments, algorithm 630 performs an analysis based on two, three, four, five, six, or all of: patient medication information (e.g. as described herein in reference to FIG. 3); patient allergy data (e.g. as described herein in reference to FIG. 4); patient diagnostic device data 850 (e.g. as described herein in reference to FIG. 5); recent patient activity (e.g. as estimated by system 10 as described herein in reference to FIG. 6, and/or as described herein in reference to FIG. 7); a previously provided FPD 70b and/or other FP 70 (e.g. as described herein in reference to FIG. 8); a patient preference (e.g. as described herein in reference to FIG. 9); and/or data 650 that has been modified via monitoring of public information by system 10.

An optional Step 10090 can be performed, in which system 10 further provides additional FP 70, which can include additional FPD 70b (e.g. in addition to what was provided in Step 10030) and/or actual food product to be ingested, IFP 70a, such as is described herein in reference to Step 2090 of FIG. 2.

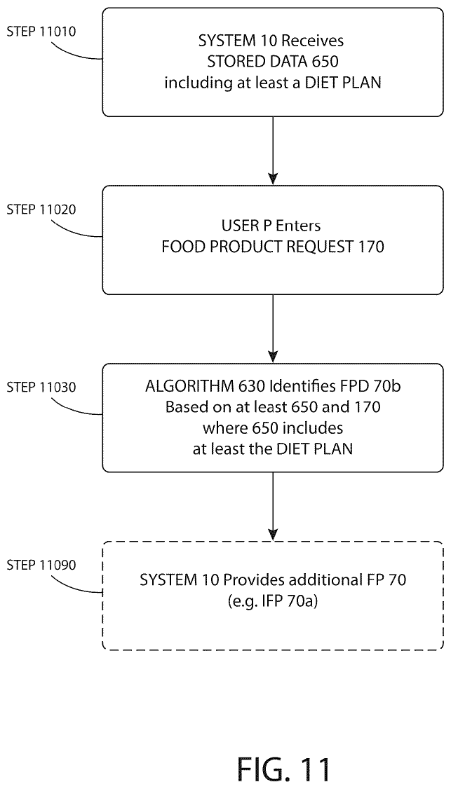

Referring now to FIG. 11, a flow chart of a method for a patient to obtain a food product based on a diet plan is illustrated, consistent with the present inventive concepts. In Step 11010, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years. In the embodiment of FIG. 11, stored data 650 comprises patient information 651 including a diet plan for the patient. The diet plan can be entered via user P using PDD 100, via a user C using CDD 200, or via another data device of system 10.

The diet plan can include food products to include and/or avoid in FP 70 provided by system 10. The diet plan can include target levels of one or more FP 70 (e.g. ingredients, calories, fat content, vitamin content, mineral content, sugar content, toxin content, and the like). The diet plan can include a diet plan made available publicly (e.g. via the internet or a printed publication), such as a publicly known ketogenic diet, low carbohydrate diet, vegetarian diet, vegan diet, raw food diet, and the like. In some embodiments, a diet plan is made available to user P (e.g. downloadable free or for a purchase price via the internet), and the diet plan can be provided to system 10 and stored as generic clinical data 652. In some embodiments, the diet plan is entered by user P, and subsequently confirmed by a user C (e.g. confirmed by a clinician of user P, such as is described herein in reference to Step 10012 of FIG. 10). In these embodiments, without confirmation, the diet plan is not implemented by system 10. In some embodiments, the clinician can modify a diet plan provided by user P. In these embodiments, a confirmation step can be included in which user P, user C, and/or another user U needs to confirm the modified diet plan prior to its implementation by system 10. As described herein, in some embodiments, more than one user U is required to confirm a modification.

In Step 11020, user P enters a food product request, FPR 170, such as by entering data into user interface 110 of PDD 100, such as is described herein in reference to Step 2020 of FIG. 2.

In Step 11030, algorithm 630 analyzes the FPR 170 and the stored data 650, and identifies FP 70 based on the analysis. System 10 can provide to user P one or more FPs 70 or suggestions for FPs 70 based on the analysis by algorithm 630, such as is described herein in reference to Step 2030 of FIG. 2. In the embodiment of FIG. 11, the analysis performed by algorithm 630 is based on the diet plan entered in Step 11010.

In some embodiments, algorithm 630 performs an analysis based on two, three, four, five, six, seven, or all of: patient medication information (e.g. as described herein in reference to FIG. 3); patient allergy data (e.g. as described herein in reference to FIG. 4); patient diagnostic device data 850 (e.g. as described herein in reference to FIG. 5); recent patient activity (e.g. as estimated by system 10 as described herein in reference to FIG. 6, and/or as described herein in reference to FIG. 7); a previously provided FPD 70b and/or other FP 70 (e.g. as described herein in reference to FIG. 8); a patient preference (e.g. as described herein in reference to FIG. 9); data 650 that has been modified via monitoring of public information by system 10 (e.g. as described herein in reference to FIG. 10); and/or a diet plan for user P.

An optional Step 11090 can be performed, in which system 10 further provides additional FP 70, which can include additional FPD 70b (e.g. in addition to what was provided in Step 11030) and/or actual food product to be ingested, IFP 70a, such as is described herein in reference to Step 2090 of FIG. 2.

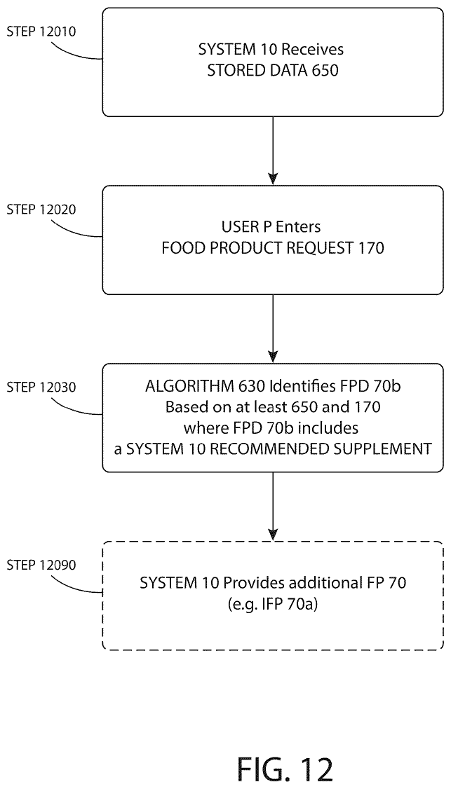

Referring now to FIG. 12 a flow chart of a method for a patient to obtain a food product that includes a system-recommended supplement is illustrated, consistent with the present inventive concepts. In Step 12010, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years.

In Step 12020, user P enters a food product request, FPR 170, such as by entering data into user interface 110 of PDD 100, such as is described herein in reference to Step 2020 of FIG. 2.

In Step 12030, algorithm 630 analyzes the FPR 170 and the stored data 650, and identifies FP 70 based on the analysis. System 10 can provide to user P one or more FPs 70 or suggestions for FPs 70 based on the analysis by algorithm 630, such as is described herein in reference to Step 2030 of FIG. 2. In the embodiment of FIG. 12, the analysis performed by algorithm 630 is configured to identify any “supplement food products” to be included in FP 70. For example, a supplement food product included in FP 70 can include a neutralizing agent, as described herein, such as to neutralize undesired substances included in a food previously ingested or to be ingested by user P. For example, FP 70 can include a neutralizing agent comprising a chelating agent, such as when FP 70 or other substance ingested by user P includes a substance known or suspected of including a metal, toxin, and/or other undesired substance (e.g. fish or other food including lead, mercury, iron, and/or arsenic). Typical chelating agents include sulfur rich foods (e.g. onions, garlic, cauliflower, eggs, brussels sprouts, and/or cabbage), sea vegetables, cilantro, chlorella, complete amino acids, and/or pectin. In some embodiments, algorithm 630 is configured to identify the timing of ingestion of one or more chelating or other neutralizing agents, as well as the amount of the neutralizing agent to be ingested (e.g. based on the amount of toxins ingested by user P).

In some embodiments, algorithm 630 performs an analysis based on two, three, four, five, six, seven, eight, or all of: patient medication information (e.g. as described herein in reference to FIG. 3); patient allergy data (e.g. as described herein in reference to FIG. 4); patient diagnostic device data 850 (e.g. as described herein in reference to FIG. 5); recent patient activity (e.g. as estimated by system 10 as described herein in reference to FIG. 6, and/or as described herein in reference to FIG. 7); a previously provided FPD 70b and/or other FP 70 (e.g. as described herein in reference to FIG. 8); a patient preference (e.g. as described herein in reference to FIG. 9); data 650 that has been modified via monitoring of public information by system 10 (e.g. as described herein in reference to FIG. 10); a diet plan for user P (e.g. as described herein in reference to FIG. 11); and/or an analysis of FP 70 for potential supplemental food products to be included.

An optional Step 12090 can be performed, in which system 10 further provides additional FP 70, which can include additional FPD 70b (e.g. in addition to what was provided in Step 12030) and/or actual food product to be ingested, IFP 70a, such as is described herein in reference to Step 2090 of FIG. 2.

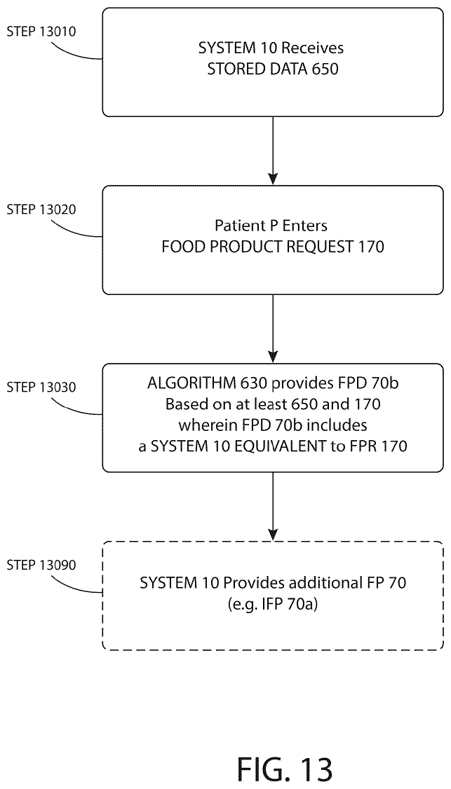

Referring now to FIG. 13, a flow chart of a method for a patient to obtain a replacement food product is illustrated, consistent with the present inventive concepts. In Step 13010, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years.

In Step 13020, user P enters a food product request, FPR 170, such as by entering data into user interface 110 of PDD 100, such as is described herein in reference to Step 2020 of FIG. 2. The FPR 170 of Step 13020 can include a request for a relatively specific food product, such as “a vanilla milkshake”, “potato chips”, “pepperoni pizza”, “coconut ice cream”, “pancakes with maple syrup”, and the like.

In Step 13030, algorithm 630 analyzes the FPR 170 and the stored data 650, and identifies FP 70 based on the analysis. System 10 can provide to user P one or more FPs 70 or suggestions for FPs 70 based on the analysis, such as is described herein in reference to Step 2030 of FIG. 2. In the embodiment of FIG. 13, the analysis performed by algorithm 630 can simply provide an exact or reasonable equivalent to the specific food product requested. However, in other embodiments, algorithm 630 provides a substitute food product, such as a healthier option (e.g. an option of similar taste, similar texture, similar presentation, and/or other similar characteristic), which algorithm 630 identifies to be a reasonable substitute for the patient, such as a healthier option to a vanilla milkshake. In some embodiments, stored data 650 includes substitute food product information received from user P, other patients using system 10, and/or other users of system 10. For example, stored data 650 can include substitutes to certain food products (e.g. milkshakes, desserts, and/or other high caloric meals) which user P or previous users of system 10 have had a positive experience ingesting (e.g. were pleased with the particular substitution). In some embodiments, algorithm 630 comprises a learning algorithm, such as an algorithm that modifies substitute food products or performs other modifications over time, based on feedback from user P, any user U, or otherwise.

In some embodiments, algorithm 630 performs an analysis based on two, three, four, five, six, seven, eight, nine, or all of: patient medication information (e.g. as described herein in reference to FIG. 3); patient allergy data (e.g. as described herein in reference to FIG. 4); patient diagnostic device data 850 (e.g. as described herein in reference to FIG. 5); recent patient activity (e.g. as estimated by system 10 as described herein in reference to FIG. 6, and/or as described herein in reference to FIG. 7); a previously provided FPD 70b and/or other FP 70 (e.g. as described herein in reference to FIG. 8); a patient preference (e.g. as described herein in reference to FIG. 9); data 650 that has been modified via monitoring of public information by system 10 (e.g. as described herein in reference to FIG. 10); a diet plan for user P (e.g. as described herein in reference to FIG. 11); an analysis of FP 70 for potential supplemental food products to be included (e.g. as described herein in reference to FIG. 12); and/or substitute food product data.

An optional Step 13090 can be performed, in which system 10 further provides additional FP 70, which can include additional FPD 70b (e.g. in addition to what was provided in Step 13030) and/or actual food product to be ingested, IFP 70a, such as is described herein in reference to Step 2090 of FIG. 2.

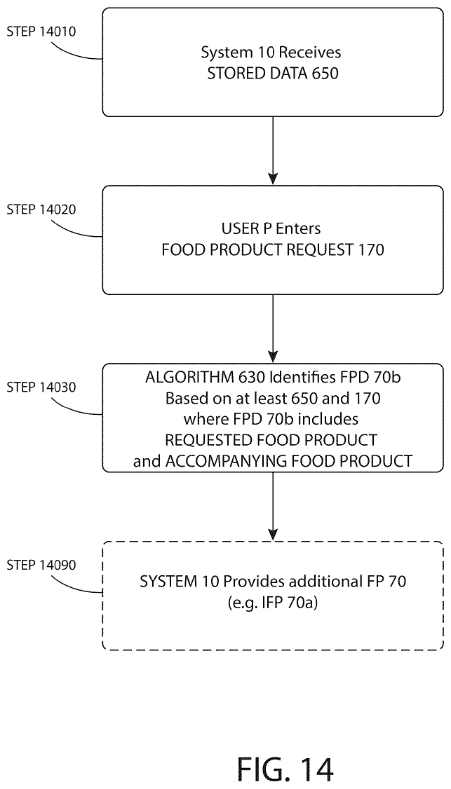

Referring now to FIG. 14, a flow chart of a method for a patient to obtain a requested food product and an additional food product is illustrated, consistent with the present inventive concepts. In Step 14010, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years.

In Step 14020, user P enters a food product request, FPR 170, such as by entering data into user interface 110 of PDD 100, such as is described herein in reference to Step 2020 of FIG. 2. The FPR 170 of Step 14020 can include a request for a relatively specific food product, such as “turmeric”, “sushi”, “something sweet”, or other specific one or more food products.

In Step 14030, algorithm 630 analyzes the FPR 170 and the stored data 650, and identifies FP 70 based on the analysis. System 10 can provide to user P one or more FPs 70 or suggestions for FPs 70 based on the analysis by algorithm 630, such as is described herein in reference to Step 2030 of FIG. 2. In the embodiment of FIG. 14, the analysis performed by algorithm 630 provides FP 70 comprising both the requested food product (e.g. or a substitute food product such as is described herein in reference to FIG. 13), as well as a suggested additional second food product (an “accompanying food product”). For example, the accompanying food product can comprise a food product that should be ingested concurrent or at least temporally proximate the ingestion of the requested food product. For example, the requested food product can comprise a food product that system 10 identifies should be included with the accompanying food product in order to achieve a desired health benefit and/or to avoid an undesired health state or undesired health risk. For example, the requested food product can comprise a substance that is difficult to be absorbed by the gastrointestinal (GI) system of the patient, and the accompanying food product can comprise a substance that helps with that absorption (e.g. fats, oils, and/or pepper that is provided to improve the absorption of turmeric). In another example, the requested food product can comprise a substance that includes (or potentially includes) one or more toxins, and the accompanying food product can comprise a substance that helps the GI system to remove those toxins (e.g. one or more chelating agents that is provided to remove mercury or other undesired substance from ingested sushi or other fish product).

In some embodiments, algorithm 630 performs an analysis based on two, three, four, five, six, seven, eight, nine, ten, or all of: patient medication information (e.g. as described herein in reference to FIG. 3); patient allergy data (e.g. as described herein in reference to FIG. 4); patient diagnostic device data 850 (e.g. as described herein in reference to FIG. 5); recent patient activity (e.g. as estimated by system 10 as described herein in reference to FIG. 6, and/or as described herein in reference to FIG. 7); a previously provided FPD 70b and/or other FP 70 (e.g. as described herein in reference to FIG. 8); a patient preference (e.g. as described herein in reference to FIG. 9); data 650 that has been modified via monitoring of public information by system 10 (e.g. as described herein in reference to FIG. 10); a diet plan for user P (e.g. as described herein in reference to FIG. 11); an analysis of FP 70 for potential supplemental food products to be included (e.g. as described herein in reference to FIG. 12); substitute food product data (e.g. as described herein in reference to FIG. 13); and/or additional food product data.

An optional Step 14090 can be performed, in which system 10 further provides additional FP 70, which can include additional FPD 70b (e.g. in addition to what was provided in Step 14030) and/or actual food product to be ingested, IFP 70a, such as is described herein in reference to Step 2090 of FIG. 2.

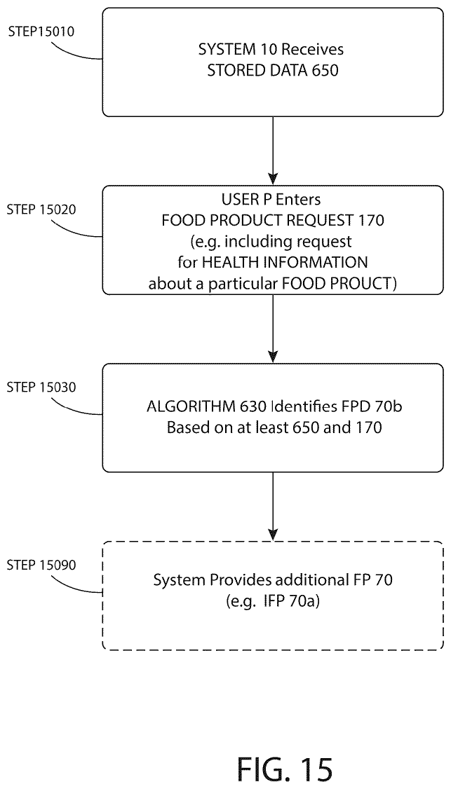

Referring now to FIG. 15, a flow chart of a method for a patient to obtain food product data regarding a specific food product is illustrated, consistent with the present inventive concepts. In Step 15010, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years.

In Step 15020, user P enters a food product request, FPR 170, such as by entering data into user interface 110 of PDD 100, such as is described herein in reference to Step 2020 of FIG. 2. The FPR 170 of Step 15020 can include a request for information regarding a specific food product, and the requested information can be provided by system 10 as FPD 70b. For example, user P can desire to receive health information about a particular food product. In some embodiments, user P transmits information to system 10, such as via PDD 100. The transferred information can be: spoken word (e.g. as recorded by a microphone of PDD 100); entered text (e.g. as recorded by a keyboard or touchscreen of PDD 100); a visual image (e.g. as captured by a camera of PDD 100). For example, user P can enter a web site address containing one or more food products. User P can take a picture of a food product (e.g. when in a grocery store). In some embodiments, information for two or more different food products are entered into system 10 by user P, such that a comparison of the two or more products can be performed in Step 15030 described herein.

In Step 15030, algorithm 630 analyzes the FPR 170 and the stored data 650, and identifies FP 70 based on the analysis. System 10 can provide to user P one or more FPs 70 or suggestions for FPs 70 based on the analysis by algorithm 630, such as is described herein in reference to Step 2030 of FIG. 2. For example, FPD 70b can comprise a quantitative or qualitative assessment of the health benefits and/or health risks of the requested one or more food products to be assessed, such as by providing a health score as defined herein. In some embodiments, the analysis performed by algorithm 630 provides a recommendation for one or more FP 70 comprising alternative food products, based on the food product for which information is requested. For example, algorithm 630 can provide a substitute food product that is identified to be similar to the requested food product (e.g. similar in taste) but has a more desirable health score.

In some embodiments, algorithm 630 performs an analysis based on two, three, four, five, six, seven, eight, nine, ten, eleven, or all of: patient medication information (e.g. as described herein in reference to FIG. 3); patient allergy data (e.g. as described herein in reference to FIG. 4); patient diagnostic device data 850 (e.g. as described herein in reference to FIG. 5); recent patient activity (e.g. as estimated by system 10 as described herein in reference to FIG. 6, and/or as described herein in reference to FIG. 7); a previously provided FPD 70b and/or other FP 70 (e.g. as described herein in reference to FIG. 8); a patient preference (e.g. as described herein in reference to FIG. 9); data 650 that has been modified via monitoring of public information by system 10 (e.g. as described herein in reference to FIG. 10); a diet plan for user P (e.g. as described herein in reference to FIG. 11); an analysis of FP 70 for potential supplemental food products to be included (e.g. as described herein in reference to FIG. 12); substitute food product data (e.g. as described herein in reference to FIG. 13); additional food product data (e.g. as described herein in reference to FIG. 14); and/or food product assessment data.

An optional Step 15090 can be performed, in which system 10 further provides additional FP 70, which can include additional FPD 70b (e.g. in addition to what was provided in Step 15030) and/or actual food product to be ingested, IFP 70a, such as is described herein in reference to Step 2090 of FIG. 2. In some embodiments, the FP 70 provided is a substitute food product.

In some embodiments, system 10 is configured as a multi-patient system, such as when multiple PDDs 100 are provided to multiple users P. System 10 can comprise different patient data 651 for each user P. All, or at least a portion, of each of generic clinical data 652, supplier data 653, and other data 654 can be “shared” among each of the users P. For example, algorithm 630 can be configured to identify an FPD 70b for a first user P′, based on a patient data 651′for that user P′, as well as information from the shared generic clinical data 652, shared supplier data 653, and shared other data 654. Algorithm 630 can be further configured to identify an FPD 70b for additional users (e.g. a second user P″, a third user P′″, and/or other users P) based on each of those user P's specific patient data 651 (e.g. patient data 651″ for second user P″, patient data 651′41 for third user P′″, and so on), as well as information from the shared generic clinical data 652, shared supplier data 653, and shared other data 654.

In some embodiments, system 10 is configured to create an estimated meal history. For example, algorithm 630 can be configured to produce the estimated meal history based on one or more of:

FPD 70b previously identified by algorithm 630; FP 70 provided by system 10 (e.g. IFP 70a and/or FPD 70b provided by system 10); user P entered information related to food products ingested; and/or system 10 detected food products ingested (e.g. detected via a sensor of system 10 and/or PDxD 800). In these embodiments, system 10 can include a confirmation routine in which user P or another user U of system 10 confirms the ingestion of one or more food products, prior to its inclusion in the estimated meal history (e.g. a food product that is detected by system 10 as having been ingested, and/or an FP 70 provided by system 10 that may or may not have actually been ingested by user P). In some embodiments, quantities of ingestion of each food product are also included in the estimated meal history (e.g. relative portion size). In some embodiments, the estimated meal history produced by system 10 simply includes categories of food products delivered, such as protein, carbohydrate, dessert, dairy, meat, fish, poultry, and the like. In some embodiments, system 10 is configured to allow the estimated meal history to be edited, such as an edit performed by user P. In some embodiments, algorithm 630 is biased to assume that FP 70 provided by system 10 was ingested by user P.

In some embodiments, PU 600 and/or another component of system 10 includes a real time clock that allows time of day and calendar information to be recorded (e.g. and included with diary information such as diary information including food product ingestion information and/or FP 70 provided information). PU 600 can be configured as an alarm clock configured to alert a user U (e.g. user P) to perform an event, such as to exercise, take a medication, and/or ingest an FP 70.

In some embodiments, PU 600 is configured to produce one or more reports, such as reports that are provided in visual, audio, and/or tangible (e.g. paper) form.

In some embodiments, algorithm 630 is configured to maintain a certain level of a substance in user P's system (e.g. cardiovascular system, neurological system, gastrointestinal system, and/or other biological system of user P). For example, algorithm 630 can be configured to identify FPD 70b to maintain a certain level of known, or at least suspected, anti-inflammatory and/or anti-cancer agents in the patient's system.

In some embodiments, system 10 is configured such that user P or other user U can enter a “recording mode” (e.g. by activating a button or other control of user interface 110 or other user interface of system 10). In the recording mode, data is recorded, such as audio data, visual data, and/or video data. For example, the recorded data can represent: an image of a product provided by a food supplier; and/or an audio, image, or video representation of a food product provided during a media event (e.g. a radio or television broadcast in which one or more food products are prepared or at least discussed). Once the data is recorded, algorithm 630 can produce FPD 70b related to the recorded data, such as an assessment of the related food products, a recipe for the related food products, and/or a location to procure the related food products. Alternatively or additionally, system 10 can provide FP 70 representing the related food products.

In some embodiments, system 10 is configured to record “food diary data” representing food products known or estimated to be ingested by user P. In these embodiments, system 10 can be configured to also record diagnostic data of the patient (e.g. via PDxD 800), and correlate changes in the diagnostic data with the food listed in the food diary as having been ingested (a temporal correlation). For example, system 10 can be configured to produce “health-food correlation data” that can include an improvement in health (as represented in the diagnostic data) associated with ingestion of certain food products and/or a decline in health (as represented in the diagnostic data) associated with ingestion of other certain food products. This health-food correlation data can be provided to a clinician of user P. This health-food correlation data can be used by algorithm 630 in identifying FP 70 for user P (e.g. to promote good health via eating of the identified FP 70 that have been determined to correlate with improvement in user P's health). In some embodiments, one or more food products are associated with an improvement in a particular medical condition, and system 10 is configured to recommend that food product to other users of system 10 (e.g. in a communal-learning arrangement).

In some embodiments, system 10 is configured to apply a fee to one or more suppliers of FP 70. For example, in a first step, system 10 receives various data (e.g. patient provided data 150, clinician provided data 250, supplier provided data 350, system manufacturer provided data 450, diagnostic device-provided data 850, therapeutic device-provided data 950, and/or other data), which is stored by PU 600 as stored data 650. Stored data 650 can comprise data collected over minutes, hours, months, and/or years. In a second step, user P enters a food product request, FPR 170, such as by entering data into user interface 110 of PDD 100. In a third step, algorithm 630 analyzes the FPR 170 and the stored data 650, and system 10 can provide to user P food product FP 70 comprising FPD 70b (e.g. FPD 70b identified by algorithm 630). FPD 70b can include a description of one or more FPs 70 to be ingested by user P. FPD 70b can be provided to user P via user interface 110 of PDD 100 or via any display or printed matter. In some embodiments, system 10 first provides a list of one or more suggested FPs 70 identified by algorithm 630, and user P selects all or a subset of the suggested FPs 70. Subsequently, system 10 provides FPD 70b to user P based on the selection. In an optional step, system 10 further provides additional FP 70, which can include additional FPD 70b (e.g. in addition to what was provided in the third step), and/or include actual food product to be ingested, IFP 70a. IFP 70a can be provided by a delivery service, or by a supplier of IFP 70a (e.g. a restaurant or grocery store). In this configuration, one or more fees can be paid to the manufacturer of system 10 by one or more suppliers of the recommended or at least provided FP 70.

In some embodiments, system 10 is configured to operate in a closed looped mode. For example, in a first step, user P ingests an FP 70, such as an FP 70 suggested or otherwise provided by system 10 (e.g. via identification by algorithm 630). In a second step, “ingestion information” is recorded by system 10, such as information related to: a patient assessment of liking or not liking the FP 70 (e.g. liking or not liking the taste of FP 70); results of a physiologic test (e.g. a blood test) performed relatively soon after the ingestion of the FP 70; a patient qualitative assessment of how the patient felt after ingestion (e.g. a negative assessment such as noting an upset stomach or other discomfort); and/or other ingestion information. The ingestion information is stored as stored data 650. In a third step, user P enters a food product request, FPR 170, such as by entering data into user interface 110 of PDD 100. In a fourth step, algorithm 630 analyzes the FPR 170 and the stored data 650 (including the ingestion information described herein), and system 10 can provide to user P food product FP 70 comprising FPD 70b that is based on the ingestion information gathered previously (e.g. as identified by algorithm 630, such as when FP 70 avoids foods that the patient didn't like, that caused an undesired physiologic response, and/or that caused patient discomfort). FPD 70b can include a description of one or more FPs 70 to be ingested by user P. FPD 70b can be provided to user P via user interface 110 of PDD 100 or via any display or printed matter. In some embodiments, system 10 first provides a list of one or more suggested FPs 70 determined (i.e. identified) by algorithm 630 (e.g. each based on the ingestion information), and user P selects all or a subset of the suggested FPs 70. Subsequently, system 10 provides FPD 70b to user P based on the selection. In an optional step, system 10 further provides additional FP 70, which can include additional FPD 70b (e.g. in addition to what was provided in the fourth step), and/or include actual food product to be ingested, IFP 70a, each based on the ingestion information.

In some embodiments, system 10 is configured to provide a rewards program for a patient and/or other user of system 10. For example, in a first step, system 10 receives recorded stored data 650, as described herein. In a second step, user P enters a food product request, FPR 170, such as by entering data into user interface 110 of PDD 100. In a third step, algorithm 630 analyzes the FPR 170 and the stored data 650, and system 10 can provide to user P food product FP 70 comprising FPD 70b that is based on the analysis of the algorithm (e.g. the FPD 70b identified by algorithm 630). FPD 70b can include a description of one or more FPs 70 to be ingested by user P. FPD 70b can be provided to user P via user interface 110 of PDD 100 or via any display or printed matter. In some embodiments, system 10 first provides a list of one or more suggested FPs 70 identified by algorithm 630, and user P selects all or a subset of the suggested FPs 70. Subsequently, system 10 provides FPD 70b to user P based on the selection. In an optional step, system 10 further provides additional FP 70, which can include additional FPD 70b (e.g. in addition to what was provided in the third step), and/or include actual food product to be ingested, IFP 70a, each based on the ingestion information. In these embodiments, system 10 can assign “points” or other quantitative or qualitative measures of use of system 10 (“points” herein), such as points awarded that correspond to frequency of repeating of the described steps to obtain additional IFP 70a and/or FPD 70b. The points can be allotted to the associated user P or other associated user of system 10 (e.g. a supplier-based user S, and/or a clinician-based user C). System 10 can be configured to allow the redemption of the collected points, such as to provide cash rewards or discounts to one or more fees (e.g. similar to an airline frequent flyer program). Points can be awarded based on the cost of FP 70 delivered to user P. Points can be awarded based on the particular supplier (e.g. a user S that provides or “sponsors” the points awarded), these points associated with FP 70 delivered to a user P from that particular supplier. Points can be awarded by system 10 based on a health score, such as when points (or more points) are awarded for healthier options of FP 70 (e.g. FP 70 that is determined by system 10 to be a healthier option specifically for the particular user P).

In some embodiments, user P enters an FPR 170 that includes at least “meal preparation request information”. Algorithm 630 can identify FPD 70b based on at least the meal preparation request information. Meal preparation request information can include information related to: type of food product (e.g. prepared food, ingredients for meal to be prepared, and the like); location of ingestion of meal; timing of ingestion of meal (e.g. within a certain time period); location of preparation of meal (e.g. within a certain distance from current location of user P); and/or food preparer description (e.g. restaurant, food delivery service, and/or self-cooked or otherwise home cooked). For example, meal preparation request information can include information similar to one or more of the following: “eat food product at restaurant”; “eat food product at restaurant close to me”; “have food product delivered to my house”; “have ingredients for food product delivered to my house within X days”; “have food product delivered to my house within XX minutes”; “cook food product myself”; “cook food product myself based on items currently in my house”. In some embodiments, algorithm 630 identifies an FPD 70b based on timing (e.g. delivered to my house within XX minutes and/or at a restaurant within XX minutes of my current location), where algorithm 630 accounts for traffic (e.g. when system 10 imports traffic information from one or more traffic-providing web services).

In some embodiments, algorithm 630 identifies an FPD 70b based on at least “patient location information”, such as the current location and/or a future location of user P. The current location of user P can be entered manually by user P (e.g. via PDD 100) or automatically determined by a GPS-based sensor of system 10 (as described herein). The future location of user P can be entered manually by user P (e.g. via PDD 100) and/or estimated by system 10 (e.g. by algorithm 630 using GPS and/or other information).

In some embodiments, system 10 is configured to provide multiple food products for selection by user P in a “menu format”. In these embodiments, algorithm 630 identifies an FPD 70b comprising the multiple food products, such as multiple food products displayed graphically on PDD 100, such that user P can select one or more of the multiple food products to be delivered to the patient as FPD 70b and/or IFP 70a. The menu format can include an assessment for one or more (e.g. each) of the displayed food products, such as an assessment that includes a health score, or includes other information (e.g. caloric content, and/or other nutritional information). Alternatively or additionally, the menu format can include other food product information for one or more (e.g. each) of the displayed food products, such as information selected from the group consisting of: cost of the food product; method of food product delivery; timing of delivery of food product; time to prepare the food product; location of food product; and combinations of one, two, or more of these.

Algorithm 630 can comprise a “learning algorithm”. For example, based on certain FPRs 170 entered by user P, algorithm 630 can determine that stored data 630 is missing sufficient information in order to properly identify an FPD 70b for user P. Once the insufficiency is identified, system 10 can be configured to obtain additional information (the “missing information), such as via an automated or manual search of available data (e.g. via the Internet or otherwise), and/or via queries sent to user P, or another user of system 10 (e.g. a user C, a user S, and/or a user M). The missing information can be added to stored data 650 (e.g. when confirmed adequate by a confirmation routine as described herein). Subsequently, algorithm 630 can identify FPD 70b based on at least the missing information. In some embodiments, algorithm 630 comprises a machine learning algorithm.

In some embodiments, user P comprises a first user P′ that is responsible for food products to be ingested by a second user P″. First user P′ can comprise one or more people, and second user P″ can comprise one or more people. First user P′ can comprise a caregiver of second user P″, such as a second user P″ comprising one or more individuals under the care of first user P′. First user P′ can comprise a head of a household responsible for preparing food for a family, second user P″ comprising the family (e.g. including first user P′, their spouse and/or children). First user P′ can comprise one or more people in charge of a cafeteria (e.g. a cafeteria of a school, hospital, day care facility, nursing home, rehabilitation facility, or the like), wherein second user P″ comprises the people that eat the food from the cafeteria. In each of these embodiments, algorithm 630 can be configured to identify FPD 70b and/or system 10 can be configured to provide FP 70 (e.g. IFP 70a and/or FPD 70b) for one or more second users P″ based on patient data 650 representing each of the one or more second users P″. In some embodiments, first user P′ enters FPR 170 for one or more meals to be provided by system 10. Alternatively or additionally, one or more second users P″ can enter an FPR 170. In some embodiments, first user P′ enters various information that is stored in stored data 650 and used by algorithm 630 to identify the FPD 70b.

In some embodiments, an FPD 70b identified by algorithm 630 and/or a IFP 70a or FPD 70b provided by system 10, is affected by a previously provided FP 70 to user P (e.g. previously provided by system 10). For example, system 10 can be configured to avoid user P receiving similar FPs 70 sequentially and/or within a certain time period (e.g. within a day, within 3 days, within 1 week, and/or within 2 weeks). In some embodiments, algorithm 630 is configured to avoid similar FP 70 to that provided in a certain number of sequential previously provided FPs 70 (e.g. to avoid repeating within a certain number of FP 70 provided cycles). In some embodiments, an FP 70 identified by algorithm 630 is affected by a previously provided (e.g. recently provided) FP 70 in order to: maintain a diet; maintain a similar caloric intake from time period to time period (e.g. day to day); to avoid exceeding a threshold (e.g. a calorie threshold, ingredient threshold, toxin threshold, and/or allergy threshold). In some embodiments, one or more food products ingested (e.g. recently ingested) by user P (e.g. whether or not identified by algorithm 630 or provided by system 10) affects which FP 70 is identified by algorithm 630. For example, these ingested food products can be entered into system 10 by user P, and/or system 10 can detect the ingestion of these food products (e.g. via a sensor of system 10 and/or via PDxD 800).

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments.

Furthermore, equivalents and modifications not described above may also be employed without departing from the scope of the inventive concepts, which is defined in the accompanying claims.

1. (canceled)

2. A method for providing a personalized health intervention to a patient user, the method comprising:
storing, in one or more non-transitory memory devices or databases accessible by a processing unit implementing an algorithm, patient data for the patient user, the patient data including at least genetic information of the patient user obtained from a patient device associated with the patient user;
storing, in the one or more memory devices, location information of the patient user obtained from a GPS sensor of the patient device;
storing, in the one or more memory devices, clinician-confirmed patient-specific clinical data for the patient user;
storing, in the one or more memory devices, food product information including ingredient information and health characteristics for a plurality of food products;
receiving, by the processing unit from the patient device, a food product request or a health status input from the patient user;
receiving, by the processing unit from a clinician device associated with a clinician user linked to the patient user, a clinician confirmation authorizing at least one of: an addition of data to, a deletion of data from, or an update of data in, the clinician-confirmed patient-specific clinical data for the patient user;
processing, by execution of the algorithm on the processing unit, the food product request or health status input by at least:
accessing the stored patient data for the patient user, including the genetic information of the patient user and the location information of the patient user;
accessing the stored food product information;
accessing the stored clinician-confirmed patient-specific clinical data for the patient user;
correlating, by the algorithm, the genetic information of the patient user with ingredient information of one or more food products from the food product information to identify potential contraindications or benefits of said one or more food products for the patient user;
analyzing, by the algorithm, the location information of the patient user in conjunction with the accessed patient data and the clinician-confirmed clinical data for the patient user to generate context-specific recommendations;
based on results of said correlating and said analyzing, identifying, by the algorithm, a specific food product from the one or more food products for the patient user, and outputting data representing said specific food product to the patient device of the patient user; and
in response to the specific food product being identified for the patient user, transmitting, from the processing unit, a control signal comprising data identifying the specific food product to a supplier data device associated with a supplier, thereby causing the supplier to prepare or deliver the specific food product to a current location of the patient user.

3. The method according to claim 2, wherein the algorithm is further configured to identify the specific food product based on at least one of an allergy, a food sensitivity, or a food intolerance of the patient user.

4. The method according to claim 2, further comprising obtaining data from at least one sensor associated with the patient user, wherein identifying the specific food product is further based on data recorded by the at least one sensor.

5. The method according to claim 2, wherein identifying the specific food product is further based on recent history information of the patient user, the recent history information comprising information about food recently ingested by the patient user.

6. The method according to claim 2, wherein identifying the specific food product is further based on patient preference information of the patient user.

7. The method according to claim 2, wherein identifying the specific food product is further based on monitored public data.

8. The method according to claim 2, wherein identifying the specific food product is further based on a diet plan associated with the patient user.

9. The method according to claim 2, wherein the specific food product includes a system-recommended supplement in addition to a base food product.

10. The method according to claim 2, wherein the specific food product includes a replacement food product substituted for a requested food product.

11. The method according to claim 2, wherein the specific food product comprises a requested food product and an additional food product.

12. The method according to claim 2, wherein identifying the specific food product is further based on generic clinical data.

13. The method according to claim 2, wherein identifying the specific food product is further based on supplier data.

14. The method according to claim 2, wherein identifying the specific food product is further based on food product transportation data associated with the one or more food products.

15. The method according to claim 2, wherein the patient user is associated with a plurality of patient data devices, including a first patient data device used by the patient user and a second patient data device used by an additional patient.

16. The method according to claim 2, wherein the method further comprises a patient diagnostic device configured to provide diagnostic data of the patient user to the processing unit, and wherein identifying the specific food product is further based on the diagnostic data.

17. The method according to claim 2, wherein the method further comprises a patient therapy device configured to perform a therapeutic event on the patient user and to provide therapeutic data to the processing unit, and wherein identifying the specific food product is further based on the therapeutic data.

18. The method according to claim 2, wherein the method further comprises receiving clinician-entered information from a clinician data device, and wherein identifying the specific food product is further based on the clinician-entered information.

19. The method according to claim 2, wherein the method further comprises receiving supplier-entered information from the supplier data device, and wherein identifying the specific food product is further based on the supplier-entered information.

20. The method according to claim 2, wherein the food product information comprises nutritional data for the plurality of food products, including at least one of:
caloric information, sugar information, carbohydrate information, fat information, trans fat information, protein information, or vitamin information for each food product.

21. The method according to claim 2, wherein the food product request comprises a request for a healthier alternative to a particular food.
